(19) United States
(12) Patent Application Publication
(10) Pub. No.: US 2010/0137426 A1
(43) Pub. Date: Jun. 3, 2010

(54) METHOD FOR GENERATING REFERENCE CONTROLS FOR PHARMACOGENOMIC TESTING

(75) Inventors: Michael P. Murphy, Raleigh, NC (US); L. Scott Clark, Cary, NC (US); Pamela J. Nakhle, Raleigh, NC (US); Kenneth G. Butz, Morrisville, NC (US)

Correspondence Address: INTELLECTUAL PROPERTY / TECHNOLOGY LAW, PO BOX 14329, RESEARCH TRIANGLE PARK, NC, 27709, US

(73) Assignee: PARAGON, Morrisville, NC (US)

(21) Appl. No.: 12/434,524

(22) Filed: May 1, 2009

Related U.S. Application Data
(60) Provisional application No. 61/049,557, filed on May 1, 2008.

(51) Int. Cl. A61K 31/352; C12Q 1/68; A61P 7/02

(52) U.S. Cl. 514/457; 435/6

(57) ABSTRACT

Reference controls for use with pharmacogenomic testing, and methods for their identification, preparation, and use, are disclosed. The reference controls can confirm that pharmacogenomic testing correctly identifies individuals that do or do not have the mutation of interest, in both clinical trial and patient treatment settings. The reference controls can be selected to include one or more mutations to be identified, and prescreened to confirm that they bind to one or more of the primers used in the pharmacogenomic testing. The reference controls are human genomic DNA that includes certain identified polymorphisms (mutations) of interest, ideally derived from individuals, pre-selected and optionally properly consented, which have one or more of the polymorphism(s) of interest. The reference controls can be prepared by targeted pre-screening of human patients, by examining the genotype or genetic profile of the patients, isolating cells with the desired mutation, optionally immortalizing the cells, and obtaining DNA from the cells. The prescreening of prospective donors can be targeted based on any of a number of factors, such as genes of interest, mutations within the genes of interest, and membership in a specific ethnic or disease state population. The genomic DNA can be pre-screened for its ability to be detected, using a standard pharmacogenomic test, as including a specific mutation. Examples of mutations of interest include those present in a Phase I or Phase II metabolic enzyme such as CYP2D6, CYP2C19, CYP2C9, CYP2C8, and CYP3A5, CYP3A4, CYP2A6, CYP2B6, UGT1A1, DPD, ERCC1, MDR1, ADH2, NAT1 and NAT2 or any other metabolic or disease gene.

### FIELD OF THE INVENTION

This application is generally in the field of pharmacogenomics, more particularly, in the sourcing and generation of reference controls for use in pharmacogenomic testing.

### BACKGROUND OF THE INVENTION

Pharmacogenomics is the study of inheritable traits affecting patient response to drug treatment. Differential responses to drug treatment may be due to underlying genetic polymorphisms (genetic variations sometimes called mutations) that affect drug metabolism. The use of pharmacogenomics may reduce the cost of clinical trials and improve patient therapy. By incorporating pharmacogenomics into their drug development programs, pharmaceutical companies can reduce the number of patients in clinical trials by patient stratification, avoiding drug failures, and omitting therapeutic monitoring during clinical trials. Testing patients for these genetic polymorphisms may help to prevent adverse drug reactions and facilitate appropriate drug dosing regimens.

In the clinical setting, pharmacogenomics may enable physicians to select the appropriate pharmaceutical agents, and the appropriate dosage of these agents, for each individual patient. That is, pharmacogenomics can identify those patients with the right genetic makeup to respond to a given therapy, and also can identify those patients with genetic variations in the genes that control the metabolism of pharmaceutical compounds, so that the proper dosage can be administered.

In the context of drug development, pharmacogenomics will permit pharmaceutical companies to identify clinically relevant subsets of patients that either will respond, or who will not respond, to a given therapy. While a given treatment may appear ineffective when administered to the general patient population, it may be extremely effective in a particular subset of the population. Identification of this subset may be crucial to obtaining FDA approval. Further, if pharmaceutically active agents are administered to all patients without regard to their ability to metabolize the agents, clinical data may be confounded. Pharmacogenomic testing to identify individual patients with altered metabolism can help guide the proper dosing of the agents, so that the clinical study is a true and accurate measure of the safety and efficacy of the agents.

In patient care, it is also important to identify responders and non-responders, as well as individuals with altered metabolism, so that the proper agent is administered at the proper dosage. In both the clinical trial and patient care settings, it is desirable that laboratory tests detect the patients that are most likely to be non-responders or exhibit altered metabolism.

Pharmacogenomic testing typically involves binding a primer to DNA which includes a sequence complementary to that of the primer. An amplification step, such as a polymerase chain reaction step, is then typically performed. Then, the presence or absence of the mutation is detected, for example, by use of a primer or probe. When the sequence includes a mutation of interest, and the primer is designed to bind to the mutated sequence, the pharmacogenomic screening can identify subjects that do or do not have the mutation of interest.

Upon initiation of clinical pharmacogenomic testing, all groups conducting such testing face a common dilemma. Method development and validation necessarily moves forward prior to possession of known positive results or other relevant reference points serving as reference controls. Without positive and negative controls, the initial process involves iterative developmental stages until a collection of samples is obtained and verified by a separate, independent method, e.g., by sequencing.

Further, even when testing groups have access to appropriate patient populations from which to obtain initial reference DNAs, it can be difficult to determine which subject DNAs are most appropriate for use as controls. Often, an initial screening project must be undertaken to build a “database” of patients with known genotypes. Another significant challenge is securing access to a patient population of sufficient size in order to sufficiently increase the chance that low frequency alleles (mutations) will be found during screening.

Another challenge is finding a reference control comprising an identical genetic sequence for the gene and/or mutation to be identified in unknown patient samples. Several overlapping factors, as discussed above, are indicative of the complexity of this challenge. First, random sequences, especially those relatively short in length, are often repeated hundreds or thousands of times within the three billion base pairs of the human genome. Second, on average, for every 600 to 1000 base pairs, there is a single nucleotide polymorphism (SNP) which is simply a difference in the base represented at a specific location with a predictable frequency from one group to the other. Fortunately, many of these polymorphisms have evolved as groups or clusters among various ethnic groups, making them somewhat more predictable. However, without having detailed “SNP maps” of all the possible combinations found in human populations, it is very possible that an unknown SNP can appear within a critical region of a mutation detection assay, causing either assay failure (e.g., allelic dropout, false negative) or false positive results (e.g., pseudogenes, mispriming).

As with any laboratory test, it is advantageous to use reference controls that reliably indicate that the pharmacogenomic tests are working properly. The present invention provides such reference controls, as well as methods for their identification, preparation and use.

### SUMMARY OF THE INVENTION

The present invention provides the sourcing and generation of reference controls used with pharmacogenomic testing. The reference controls described herein can be used to confirm that the pharmacogenomic testing correctly identifies individuals that do or do not have the mutation of interest. The reference controls can be selected so that they include one or more of the mutations that are to be identified, and also that they bind to one or more of the primers used in the pharmacogenomic testing. Thus, they can be used to confirm that the pharmacogenomic testing actually detects the presence of the mutation(s).

The reference controls themselves comprise human genomic DNA that include certain identified polymorphisms (mutations) of interest. The source of these controls can be lymphocytes (or other human tissue) from individuals, pre-selected and optionally properly consented, which have one or more of the polymorphism(s) of interest. Ideally, the patient screening process follows IRB-approved protocols.

Once identified and collected, the individuals' cells can be propagated and immortalized in cell lines. The cells are harvested and human genomic DNA is extracted and purified, and the individual's genotype can be confirmed by DNA re-sequencing. The reference controls provide a source of human genomic DNA that is, or can be, nearly infinite in supply. Because the reference control DNA can be propagated in human cells, the controls can have a genuine copy number of the genes of interest, ensuring that amplification efficiency is representative of native conditions.

In some embodiments, the DNA is derived from immortalized or cancer cell lines (cell lines with continuously dividing cells). The reference controls provide a continuous and reliable source of human genomic DNA with the desired gene, nucleotide sequence, or genetic rearrangements. For the genes of interest, these can include the same, or approximately the same, copy number found in normal human cells. This provides the ability to titrate the DNA copy number. The reference controls can be prepared using various methods.

The reference controls can be prepared by targeted pre-screening of properly consented normal human volunteers using a validated assay, and examining the genotype or genetic profile of the volunteers. Then, those volunteers with genotypes of interest can be recalled in order to obtain a sufficient amount of lymphocytes or other suitable cell types for preparation of immortalized cells. In a preferred embodiment, the patients/volunteers are compound heterozygotes (i.e., their genes include two or more known mutations), so that the reference controls are effective for testing for more than one mutation.

The prescreening of prospective donors can be targeted based on any of a number of factors. These include, for example, genes of interest, mutations within the genes of interest, and membership in a specific ethnic or disease state population. Also, one can consider the known frequency of the desired mutation or gene to determine how many patients or volunteers need to be tested in order to find one or more with the desired genetic profile.

In still another aspect, the reference controls are prepared by screening cell cultures commonly available for the genetic sequence of interest, and then expanding and preserving the cell clones for future continuous supply.

Once cells that have a mutation of interest are identified through a pre-screening step, such as a rapid genotyping assay, confirmation that the cells include the mutation of interest can be confirmed by a more thorough sequencing method.

The genomic DNA extracted from the cells containing a mutation of interest serve as a putative reference control. Putative reference controls can be validated using a previously validated assay, with primers known to bind to sequences that include the mutation of interest. By performing a genotyping assay using the putative reference control and the validated primer, one can validate the reference control.

The reference controls can be those in which only a single assay was used to detect the presence of the polymorphism, but ideally are those in which the presence of the polymorphism has been confirmed with a plurality of assays. The use of a plurality of assays helps ensure that the control can be used with other assay methods, and is not just validated for use with a single assay, such as the assay used to initially identify the sample as having the polymorphism of interest.

The primer sets used by diagnostic kit manufacturers (and the amplicons generated from these primers) to identify the specific mutation will, by necessity, include a specific DNA sequence several base pairs to the left and/or right of the mutation of interest. The putative reference control must bind to this primer with a certain affinity to be detected as containing the mutation of interest. Pre-screening with validated primer(s) can detect putative reference controls that not only include a mutation of interest, but which also contain one or more additional mutations. In some instances, the additional mutations would be present in the primer binding site, thus preventing the reference control from binding to the primer of interest.

The validated reference control can be used in assay development to develop new primers. Primers can be designed by selecting a piece of DNA that includes a certain number of base pairs to the left and/or right of the SNP of interest, based on the known sequence of the normal (wild type) sequence and the position of the SNP. By confirming that the primer binds to the validated reference control under the appropriate assay conditions, one can validate the primer.

The validated reference controls and validated primers can be used in assay validation, to confirm that laboratory personnel are capable of performing the assay correctly. If the assay is run with validated reference controls and validated primers, but the personnel are unable to obtain an appropriate result, then the personnel can be trained until they are able to successfully perform the assay.

In each embodiment, the cells can be immortalized, for example, using Epstein-Barr Virus (EBV) or other known methods for immortalizing cells. The immortalization of the cells, such as lymphocytes, ensures a standard, reliable, and continuous supply with a relatively stable genome comprising the gene or mutations of interest.

In a preferred embodiment, the gene of interest contains a mutation in a Phase I or Phase II metabolic enzyme, efficacy marker or drug target such as CYP2D6, CYP2C19, CYP2C9, CYP2C8, and CYP3A5, CYP3A4, CYP2A6, CYP2B6, UGT1A1, DPD, ERCC1, MDR1, ADH2, VKORC1, NAT1 and NAT2 or any other metabolic or disease gene.

The DNA isolated from the cells can be used as positive human genomic reference controls (i.e., they have mutations present) or negative controls (i.e., they represent the normal or wild-type), in particular, for human Cytochrome P450 genes. This can ensure accurate and reliable clinical diagnostic testing for these genes.

The reference controls can be used, for example, in genotyping assays performed during clinical trials. Where the reference controls include a genetic variation typical of a patient who does not respond to therapy, the use of reference controls helps ensure that the genotyping assay used performs reliably such that non-responders are properly identified and data regarding the ineffectiveness of the investigative therapy for non-responders is properly identified. Similarly, where the reference controls include a genetic variation typical of a patient who metabolizes drugs at a different rate than normal patients (i.e., patients with mutant cytochrome P450 genes), the use of reference controls helps ensure the validity of the genetic variation so that these patients are properly identified and properly dosed and adverse drug reactions or ineffective therapies are avoided.

The reference controls can also be used in patient care. As with their use in clinical trials, it is essential that effective therapy is identified in a time-sensitive manner, so that the patient's condition is not worsened before appropriate therapy is initiated. It is also essential that appropriate dosing regimens are selected.

Accordingly, one aspect of the present invention relates to a method of testing a plurality of patients for their genetic predisposition to respond to a particular therapy. In this aspect, one or more reference controls are tested as “samples”—with known expected results. These controls can include a genetic variant associated with patients who show a predisposition to not respond to the therapy (positive controls) or can include a normal/wild type variant (negative controls), associated with patients who show a predisposition to respond to the therapy.

Thus, the reference controls can be used by testing laboratories to ensure that their diagnostics assays are performing correctly and identify the genetic variations that convey resistance to drug therapy or reduced metabolic state. To ensure that non-responders are properly identified, testing labs can include reference controls in each assay to determine the validity of the assay, and hence, patient results. The reference controls can be used at random, or at pre-determined intervals. In the same respect, testing laboratories can use the reference controls as panels to evaluate the accuracy of their laboratory staff.

Accordingly, another aspect of the present invention relates to a method of testing a plurality of patients for their genetic predisposition to show rapid or slowed metabolism, so that proper dosing regimens can be set. In this aspect, one or more samples that are tested are reference controls that include a genetic variant associated with patients having a predisposition to be rapid or slow metabolizers.

The particular genes of interest, or, more particularly, mutations within genes of interest, can vary depending on the type of pharmaceutically active agent that is to be given. That is, different active agents function by interacting with different receptors, enzymes, and other biological targets. Mutations in these targets, and the mutations in the genes of interest that code for these targets, have been identified for a number of targets and the active agents that bind to and interact with these targets. Appropriate reference controls for any of these targets can be prepared using the methods described herein.

The reference controls can be included in FDA-regulated, in vitro diagnostic (“IVD”) kits, as customized human genomic reference controls. The reference controls can be tested in each assay run, or at predetermined intervals, to ensure the reliability of the kits and accuracy of the testing methods. The reference controls can also be used as external controls in conjunction with FDA-regulated, in vitro diagnostic (“IVD”) kits, or home-brew assays that are developed and validated in individual laboratories.

The methods described herein for identifying, making, and/or using the reference controls can be used by a company to manufacture a product which standardizes the process and availability of human genomic reference controls for research, clinical diagnostics, and assay validation and development. Clinical diagnostic laboratories can thus obtain human genomic reference controls on an ongoing and continuous basis with the same cells (as background), and using the same process (i.e., following GMP protocols). Because validated controls are being used, laboratories can use these controls to validate assays developed in-house, for training and proficiency testing of lab personnel and for routine quality control. The results generated can be compared to those of other labs to determine whether the results obtained by one laboratory are more reliable than another.

### BRIEF DESCRIPTION OF THE FIGURES

FIGS. 1A-D are flowcharts outlining the various steps for one embodiment of creating a validated reference control.

### DETAILED DESCRIPTION OF THE INVENTION

Using the processes described herein for producing and validating positive control cell lines and genomic DNA, sustainable implementation of these processes can alleviate the current shortage of positive reference control materials. The reference controls, and methods for making and using them, are described in more detail below.

The present invention will be better understood with reference to the following non-limiting definitions:

CLIA: The Clinical Laboratory Improvement Amendments of 1988 (CLIA) established quality standards for all laboratory testing to ensure the accuracy, reliability and timeliness of patient test results regardless of where the test was performed. A laboratory is defined as any facility that performs laboratory testing on specimens derived from humans for the purpose of providing information for the diagnosis, prevention, treatment of disease, or impairment of, or assessment of health.

A DNA sequence (sometimes genetic sequence) is a succession of letters representing the primary structure of a real or hypothetical DNA molecule or strand, The possible letters are A, C, G, and T, representing the four nucleotide subunits of a DNA strand (adenine, cytosine, guanine, thymine), and typically these are printed abutting one another without gaps, as in the sequence AAAGTCTGAC. This coded sequence is sometimes referred to as genetic information. A succession of any number of nucleotides greater than four may be called a “sequence.” With regard to its biological function, which may depend on context, a sequence may be sense or anti-sense (see DNA), and either coding or noncoding. DNA sequences can also contain “junk DNA” (portions of the DNA sequence of a chromosome or a genome for which no function has yet been identified).

EBV: Epstein Barr Virus: the herpes virus that causes infectious mononucleosis; associated with specific cancers in Africa and China. Immortalization by EBV is an effective procedure for inducing long-term growth of human B-lymphocytes. Immortalization of cells takes a few weeks following which larger amounts of cells can be grown indefinitely as a continuing source of genetic material. Thus, a few weeks from the start of the culture, cells can be supplied in a large amount as a source of DNA. Protocols for generating EBV-transformed B cell lines are commonly known in the art, such as, for example, the protocol outlined in Chapter 7.22 of Current Protocols in Immunology, Coligan et al., Eds., 1994, John Wiley & Sons, NY, which is hereby incorporated in its entirety by reference. Also see, U.S. Pat. No. 6,926,898, also hereby incorporated by reference in its entirety.

Genotype: The genetic makeup, as distinguished from the physical appearance, of an organism or a group of organisms. Also, the combination of alleles located on homologous chromosomes that determines a specific characteristic or trait.

Mutation, missense: A genetic change involving the substitution of one base in the DNA for another which results in the substitution of one amino acid in a polypeptide for another. A missense mutation is a “readable” genetic message although its “sense” (its meaning) is changed, in contrast to a nonsense mutation which has no meaning except to halt the reading of the genetic message.

Immortalized: An “immortalized” cell, as the term is used herein unless otherwise indicated, is a cultured cell that will continue to propagate in vitro for a sufficient length of time and number of generations to allow a useful supply of genomic DNA to be obtained for use according to the present invention.

Plurality: As used herein, a plurality of assays includes at least two assays, and preferably includes three or more, more preferably, four or more, and most preferably, five or more assays.

Primer: A primer is an oligonucleotide sequence of DNA that binds to the complementary DNA sequence of interest and is involved in amplification of the target sequence. A primer can be a diagnostic primer in that binding of the primer indicates the presence of the mutation.

Probe: A probe is an oligonucleotide sequence of DNA that binds to a complementary DNA sequence of interest and is involved in hybridization of the target sequence.

Primer/Probe: For this application, primer and probe, will be used interchangeably.

I. Representative Mutations that can be Present in the Reference Controls

Using the methods described herein, validated reference controls can be prepared that include any of a variety of mutations. These mutations include mutations in the genes that control metabolism, that affect the ability to respond to a particular therapy, or that are responsible for a particular disease state. Examples of these mutations are described in detail below.

A. Cytochrome P450

Cytochrome P450 (CYP450) is a family of genes which play a primary role in drug metabolism. The CYP2 and CYP3 families are merely two members of the CYP450 super family which are considered to be of particular importance. Differences in CYP450 enzyme activity affect whether a drug reaches therapeutic levels in the blood, and also how well the drug is cleared from the body. One or more mutations in an individual's CYP450 enzymes can cause the individual to respond to drug therapy in a profoundly different manner than an individual with normal CYP450 enzymes.

CYP2D6, CYP2C9 and CYP2C19 genes play an important role in the metabolism of several widely prescribed drugs. For example, CYP2D6 is involved in metabolism of several drugs used to treat severe depression, schizophrenia, cardiovascular disease (such as beta blockers), and ADHD while CYP2C19 is involved in the metabolism of anti-convulsants, proton pump inhibitors, benzodiazepines, and anti-malarials, and both are involved in the metabolism of certain tricyclic antidepressant drugs. CYP2C9 is involved in the metabolism of the more active S-enantiomer of the anticoagulant warfarin and is also responsible for the metabolism of several nonsteroidal anti-inflammatory drugs (NSAIDs). Patients whose CYP2D6, CYP2C9 or CYP2C19 enzymes extensively metabolize certain drugs are at increased risk for experiencing toxicity with standard dosing, while ultra-rapid metabolizers may not achieve therapeutic plasma concentrations of these drugs. Further, when prodrugs are administered, a poor metabolizer will not obtain a significant benefit from the drugs.

Genetic polymorphisms of drug metabolizing enzymes (e.g., cytochrome P-450 (CYP450) enzymes such as, for example, CYP2D6, CYP2A6, CYP2C8, CYP2C9, CYP2C19, CYP3A4/5, and CYP1A2) or drug target enzymes (e.g. vitamin K epoxide reductase (VKOR), or more specifically Complex I from this enzyme (VKORC1)) explain why some individuals do not obtain the expected drug effects or show exaggerated drug response and serious toxicity after taking the standard and safe dose of a drug. These polymorphisms are expressed in a number of phenotypes in the population such as, for example, the poor metabolizer (PM), the intermediate metabolizer (IM), the extensive metabolizer (EM), and the ultra-rapid metabolizer (UM). Typically, extensive metabolizers have at least one, and no more than two, normal functional alleles. Intermediate metabolizers possess one reduced activity allele and one null allele. Poor metabolizers carry two mutant alleles which result in complete loss of enzyme activity. Ultra-rapid metabolizers typically carry multiple copies (3-13) of functional alleles, and thus produce excess enzymatic activity.

The gene coding for CYP2D6 is highly polymorphic and several mutations have been identified in PMs, which all lead to the absence of functional CYP2D6. The CYP2D6 gene is part of a cluster of 3 genes arranged in tandem on chromosome 22q13.1. The CYP2D gene cluster is composed of 2 pseudogenes, CYP2D8P and CYP2D7P, as well as the CYP2D6 gene (Heim et al., Genomics 14: 49, 1992). The only functional gene present in the human CYP2D gene locus is inactive in 5-10% of Caucasian individuals because of detrimental mutations. More than 45 major polymorphic CYP2D6 alleles have been described (See http://www.imm.ki.se/CYPalleles/cyp2d6.htm). PMs frequently experience exaggerated drug response and side effects when they receive standard doses. If a metabolite is the active therapeutic moiety, PMs show no therapeutic response as exemplified by the analgesic effect of codeine mediated by its CYP2D6-formed metabolite morphine. At the other extreme, ultra-rapid metabolizers fail to respond to standard doses. Recent studies have determined that ultra-rapid metabolism can be attributable to CYP2D6 gene duplication.

Genetic variations in these genes are distributed disproportionally among people of different racial populations and/or geographical origins, with some polymorphisms and alleles found in virtually one racial or ethnic population. For example, about 7% of Caucasians have mutations in CYP2D6 resulting in poor metabolism, whereas only about 1-2% of Asians and 2-4% of African Americans have mutations that convey a PM status. However, there is a high prevalence of several “reduced activity” alleles, such as the CYP2D6*10 allele, in Asians or the CYP2D6*17 alleles in certain African populations. These “reduced activity” mutations result in intermediate metabolizers with low enzyme activity (approximately 30% for CYP2D6*10 in Asians and CYP2D6*17 in Africans). Also, roughly 20% of Ethiopians, 10% of Southern Europeans, and 1-2% of Northern Europeans carry CYP2D6 gene duplications, which often results in ultra rapid metabolism. Non-limiting examples of drugs metabolized by CYP2D6 are psychotropics such as amitriptyline, aripiprazole, benztropine, chlorpromazine, citalopram, clomipramine, desipramine, donepezil, doxepin, duloxetine, fluoxetine, fluvoxamine, haloperidol, imipramine, mirtazapine, nortripyline, paroxetine, perphenazine, risperidone, sertraline, thioridazine, venlafaxine, zuclopenthixol; antihistamines such as chlorpheniramine, diphenhydramine, hydroxyzine; l Attention Deficit Hyperactivity Disorder (ADHD) drugs such as amphetamines, atomoxetine; beta-blockers such as carvedilol, metoprolol, timilol; cardiovascular drugs such as encainide, flecainide, mexiletine, propafenone, dexfenfluramine, metoclopramide, ondansetron; cough suppressants such as dextromethorphan; opiates such as hydrocodone, tramadol, perhexiline, phenacetin, phenformin, propranolol, sparteine, ranitidine, and tolterodine.

The majority of poor CYP2C19 metabolizers result from two common alleles, CYP2C19*2 and CYP2C19*3, both of which are null alleles caused by a single polynucleotide polymorphism resulting in a splice site defect or a stop codon, respectively. The two alleles are relatively common (13-23%) in Asian populations, whereas the CYP2C19 poor metabolizer phenotype is found in about 3-5% of Caucasian and African American populations. Non-limiting examples of drugs metabolized by CYP2C19 are psychotropics such as amitriptyline, citalopram, clomipramine, diazepam, fluoxetine, imipramine, sertraline, valproate; anticonvulsants such as mephenyloin, Phenobarbital, pheytoin; proton pump inhibitors such as lansoprazole, omeprazole, pantoprazole, carisprodol, cyclophosphamide, moclobemide, nelfinavir, progesterone, nilutamide, proguanil, propranolol, ranitidine, and R-warfarin.

The majority of poor CYP2C9 metabolizers result from two alleles, CYP2C9*2 and CYP2C9*3. These polymorphisms are observed in ˜20-30% of the Caucasian population, but are rare in African American or Asian populations. Mutations within CYP2C9 may account for up to 15% of the variance in dosage within patients taking warfarin. Non-limiting examples of drugs metabolized by CYP2C9 are psychotropics such as fluoxetine, sertraline, valproate; non-steroidal anti-inflammatory drugs and COX₂ inhibitors such as ibuprofen, flurbiprofen, naproxen, diclofenac, suprofen, piroxicam, meloxicam, celecoxib; oral hypoglycemics such as glipizide, glimepiride, glyburide, nateglinide, rosiglitazone, tolbutamide; and angiotensin −2 blockers such as irbesartan, losartan, valsartan, and cyclophosphamide.

One can capture approximately 99% of the most important mutations that predict patient outcome, and categorize poor, extensive, or ultra-rapid metabolizers, by screening for mutations in CYP2D6, CYP2A6, CYP2C8, CYP2C9, CYP2C19, CYP3A4/5, and CYP1A2 in the CYP450 family, and CDA, DPD, GSTM1/GSTT1, GSTP1, MTHFR, NAT2, OPRT, TS, and UGT1A1, which are other genes associated with metabolism.

A cytochrome P450 allele website is available from Sweden at http://www.imm.ki.se/CYPalleles/, the contents of which are hereby incorporated by reference, and additional mutations suitable for inclusion in reference controls can be identified there. Examples of alleles of the CYP450 genes, and the protein in which the allele is associated, are provided below, and additional polymorphisms suitable for DNA reference controls can be identified there. Illustratively, alleles of the CYP450 genes (shown in italics), and their associated proteins (shown in non-italicized capital letters in parenthesis, where possible), include without limitation CYP1A1*1A (CYP1A1.1), CYP1A1*1B (CYP1A1.1), CYP1A1*1C (CYP1A1.1), CYP1A1*2A (CYP1A1.1), CYP1A1*2B (CYP1A1.2), CYP1A1*2C (CYP1A1.2), CYP1A1*3 (CYP1A1.1), CYP1A1*4 (CYP1A1.4), CYP1A1*5 (CYP1A1.5), CYP1A1*6 (CYP1A1.6), CYP1A1*7, CYP1A1*8 (CYP1A1.8), CYP1A1*9 (CYP1A1.9), CYP1A1*10 (CYP1A1.10), CYP1A1*11 (CYP1A1.11), CYP1A2*1A (CYP1A2.1), CYP1A2*1B (CYP1A2.1), CYP1A2*1C (CYP1A2.1), CYP1A2*1D (CYP1A2.1), CYP1A2*1E (CYP1A2.1), CYP1A2*1F (CYP1A2.1), CYP1A2*1G (CYP1A2.1), CYP1A2*1H (CYP1A2.1), CYP1A2*1J (CYP1A2.1), CYP1A2*1K (CYP1A2.1), CYP1A2*1L Predicted haplotype (CYP1A2.1), CYP1A2*1M Predicted haplotype (CYP1A2.1), CYP1A2*1N Predicted haplotype (CYP1A2.1), CYP1A2*1P Predicted haplotype (CYP1A2.1), CYP1A2*1Q Predicted haplotype (CYP1A2.1), CYP1A2*1R Predicted haplotype (CYP1A2.1), CYP1A2*1S Predicted haplotype (CYP1A2.1), CYP1A2*1T Predicted haplotype (CYP1A2.1), CYP1A2*1U Predicted haplotype (CYP1A2.1), CYP1A2*2 (CYP1A2.2), CYP1A2*3 (CYP1A2.3), CYP1A2*4 (CYP1A2.4), CYP1A2*5 (CYP1A2.5), CYP1A2*6 (CYP1A2.6), CYP1A2*7, CYP1A2*8 (CYP1A2.8), CYP1A2*9 (CYP1A2.9), CYP1A2*10 (CYP1A2.10), CYP1A2*11 (CYP1A2.11), CYP1A2*12 (CYP1A2.12), CYP1A2*13 (CYP1A2.13), CYP1A2*14 (CYP1A2.14), CYP1A2*15 (CYP1A2.15), CYP1A2*16 (CYP1A2.16), CYP1B1*/(CYP1B1.1), CYP1B1*2 (CYP1B1.2), CYP1B1*3 (CYP1B1.3), CYP1B1*4 (CYP1B1.4), CYP1B1*5 (CYP1B1.5), CYP1B1*6 (CYP1B1.6), CYP1B1*7 (CYP1B1.7), CYP1B1*11 (CYP1B1.11), CYP1B1*12 (CYP1B1.12), CYP1B1*13, CYP1B1*14, CYP1B1*1, CYP1B1*16, CYP1B1*17, CYP1B1*18 (CYP1B1.18), CYP1B1*19 (CYP1B1.19), CYP1B1*20 (CYP1B1.20), CYP1B1*21 (CYP1B1.21), CYP1B1*22, CYP1B1*23 (CYP1B1.23), CYP1B1*24, CYP1B1*25 (CYP1B1.25), CYP1B1*26, CYP2A6*1A (CYP2A6.1), CYP2A6*1B1 (formerly CYP2A6*1B, has also been called CYP2A6*1E) (CYP2A6.1), CYP2A6*1B2 (has also been called CYP2A6*1B) (CYP2A6.1), CYP2A6*1B3 (formerly CYP2A6*1C) (CYP2A6.1), CYP2A6*1B4 (CYP2A6.1), CYP2A6*1B5 Tentative allele (CYP2A6.1), CYP2A6*1B6 Predicted haplotype/Tentative allele (CYP2A6.1), CYP2A6*1B7 Predicted haplotype/Tentative allele (CYP2A6.1), CYP2A6*1B8 Tentative allele (CYP2A6.1), CYP2A6*1B9 Predicted haplotype/Tentative allele (CYP2A6.1), CYP2A6*1B10 Predicted haplotype/Tentative allele (CYP2A6.1), CYP2A6*1B11 Predicted haplotype/Tentative allele (CYP2A6.1), CYP2A6*1B12 Predicted haplotype/Tentative allele (CYP2A6.1), CYP2A6*1C, CYP2A6*1D Predicted haplotype (CYP2A6.1), CYP2A6*1E, CYP2A6*1F (CYP2A6.1), CYP2A6*1G (CYP2A6.1), CYP2A6*1H Predicted haplotype (CYP2A6.1), CYP2A6*1J Predicted haplotype (CYP2A6.1), CYP2A6*1X2 (CYP2A6.1), CYP2A6*2 (CYP2A6.2), CYP2A6*3 (CYP2A6.3), CYP2A6*4A, CYP2A6*4B, CYP2A6*4C, CYP2A6*4D, CYP2A6*5 (CYP2A6.5), CYP2A6*6 (CYP2A6.6), CYP2A6*7 (CYP2A6.7), CYP2A6*8 (CYP2A6.8), CYP2A6*9A (CYP2A6.1), CYP2A6*9B (CYP2A6.1), CYP2A6*10 (CYP2A6.10), CYP2A6*11 (CYP2A6.11), CYP2A6*12A (CYP2A6.12), CYP2A6*12B (CYP2A6.12), CYP2A6*12C (CYP2A6.12), CYP2A6*13 (CYP2A6.13), CYP2A6*14 (CYP2A6.14), CYP2A6*15 (CYP2A6.15), CYP2A6*16 (CYP2A6.16), CYP2A6*17 (CYP2A6.17), CYP2A6*18A (CYP2A6.18), CYP2A6*18B (CYP2A6.18), CYP2A6*18C Predicted haplotype (CYP2A6.18), CYP2A6*19 (CYP2A6.19), CYP2A6*20 (CYP2A6.20), CYP2A6*21 Predicted haplotype (CYP2A6.21), CYP2A6*22 Predicted haplotype (CYP2A6.22), CYP2A13*1A (CYP2A13.1), CYP2A13*1B (CYP2A13.1), CYP2A13*1C (CYP2A13.1), CYP2A13*1D (CYP2A13.1), CYP2A13*1E (CYP2A13.1), CYP2A13*1F (CYP2A13.1), CYP2A13*1G (CYP2A13.1), CYP2A13*1H (CYP2A13.1), CYP2A13*1J (CYP2A13.1), CYP2A13*1K (CYP2A13.1), CYP2A13*1L (CYP2A13.1), CYP2A13*2A (CYP2A 13.2), CYP2A13*2B (CYP2A 13.2), CYP2A13*3 (CYP2A 13.3), CYP2A13*4 (CYP2A13.4), CYP2A13*5 (CYP2A13.5), CYP2A13*6 (CYP2A13.6), CYP2A13*7 (CYP2A 13.7), CYP2A13*8 (CYP2A 13.8), CYP2A13*9 (CYP2A 13.9), CYP2B6*1A (CYP2B6.1), CYP2B6*1B (CYP2B6.1), CYP2B6*1C (CYP2B6.1), CYP2B6*1D (CYP2B6.1), CYP2B6*1E (CYP2B6.1), CYP2B6*1F (CYP2B6.1), CYP2B6*1G (CYP2B6.1), CYP2B6*1H (CYP2B6.1), CYP2B6*1J (CYP2B6.1), CYP2B6*1K (CYP2B6.1), CYP2B6*1L (CYP2B6.1), CYP2B6*1M (CYP2B6.1), CYP2B6*1N (CYP2B6.1), CYP2B6*2A (CYP2B6.2), CYP2B6*2B (CYP2B6.2), CYP2B6*3 (CYP2B6.3), CYP2B6*4A (CYP2B6.4), CYP2B6*4B (CYP2B6.4), CYP2B6*4C (CYP2B6.4), CYP2B6*4D (CYP2B6.4), CYP2B6*5A (CYP2B6.5), CYP2B6*5B (CYP2B6.5), CYP2B6*5C (CYP2B6.5), CYP2B6*6A (CYP2B6.6), CYP2B6*6B (CYP2B6.6), CYP2B6*6C (CYP2B6.6), CYP2B6*7A (CYP2B6.7), CYP2B6*7B (CYP2B6.7), CYP2B6*8 (CYP2B6.8), CYP2B6*9 (CYP2B6.9), CYP2B6*10 (CYP2B6.10), CYP2B6*11A (CYP2B6.11), CYP2B6*11B (CYP2B6.11), CYP2B6*12 Tentative allele (CYP2B6.12), CYP2B6*13A (CYP2B6.13), CYP2B6*13B (CYP2B6.13), CYP2B6*14 Tentative allele (CYP2B6.14), CYP2B6*15A (CYP2B6.15), CYP2B6*15B (CYP2B6.15), CYP2B6*16 (CYP2B6.16), CYP2B6*17A (CYP2B6.17), CYP2B6*17B (CYP2B6.17), CYP2B6*18 (CYP2B6.18), CYP2B6*19 (CYP2B6.19), CYP2B6*20 (CYP2B6.20), CYP2B6*21 (CYP2B6.21), CYP2B6*22 (CYP2B6.1), CYP2B6*23 (CYP2B6.23), CYP2B6*24 (CYP2B6.24), CYP2B6*25 (CYP2B6.25), CYP2C8*1A (CYP2C8.1), CYP2C8*1B (CYP2C8.1), CYP2C8*1C (CYP2C8.1), CYP2C8*2 (CYP2C8.2), CYP2C8*3 (CYP2C8.3), CYP2C8*4 (CYP2C8.4), CYP2C8*5, CYP2C8*6 (CYP2C8.6), CYP2C8*7 CYP2C8*8 (CYP2C8.8), CYP2C8*9 (CYP2C8.9), CYP2C8*10 (CYP2C8.10), CYP2C9*1A (CYP2C9.1), CYP2C9*1B Predicted haplotype (CYP2C9.1), CYP2C9*1C Predicted haplotype (CYP2C9.1), CYP2C9*1D Predicted haplotype (CYP2C9.1), CYP2C9*2A Predicted haplotype (CYP2C9.2), CYP2C9*2B Predicted haplotype (CYP2C9.2), CYP2C9*2C Predicted haplotype (CYP2C9.2), CYP2C9*3A Predicted haplotype (CYP2C9.3), CYP2C9*3B Predicted haplotype (CYP2C9.3), CYP2C9*4 (CYP2C9.4), CYP2C9*5 (CYP2C9.5), CYP2C9*6, CYP2C9*7 (CYP2C9.7), CYP2C9*8 (CYP2C9.8), CYP2C9*9 (CYP2C9.9), CYP2C9*10 (CYP2C9.10), CYP2C9*11A Predicted haplotype (CYP2C9.11), CYP2C9*11B Predicted haplotype (CYP2C9.11), CYP2C9*12 (CYP2C9.12), CYP2C9*13 (CYP2C9.13), CYP2C9*14 (CYP2C9.14), CYP2C9*15 (CYP2C9.15), CYP2C9*16 (CYP2C9.16), CYP2C9*17 (CYP2C9.17), CYP2C9*18 (CYP2C9.18), CYP2C9*19 (CYP2C9.19), CYP2C9*20 (CYP2C9.20), CYP2C9*21 (CYP2C9.21), CYP2C9*22 (CYP2C9.22), CYP2C9*23 (CYP2C9.23), CYP2C9*24 (CYP2C9.24), CYP2C19*1A (CYP2C19.1A), CYP2C19*1B (CYP2C19.1B), CYP2C19*1C (CYP2C19.1B), CYP2C19*2A, CYP2C19*2B, CYP2C19*2C (also called CYP2C19*21), CYP2C19*3A, CYP2C19*3B (also called CYP2C19*20), CYP2C19*4, CYP2C19*5A (CYP2C19.5A), CYP2C19*5B (CYP2C19.5B), CYP2C19*6 (CYP2C19.6), CYP2C19*7, CYP2C19*8 (CYP2C19.8), CYP2C19*9 (CYP2C19.9), CYP2C19*10 (CYP2C19.10), CYP2C19*11 (CYP2C19.11), CYP2C19*12 (CYP2C19.12), CYP2C19*13 (CYP2C19.13), CYP2C19*14 (CYP2C19.14), CYP2C19*15 (CYP2C19.15), CYP2C19*16 (CYP2C19.16), CYP2C19*1, CYP2C19*18 (CYP2C19.18), CYP2C19*19 (CYP2C19.19), CYP2C19*20, CYP2C19*21, CYP2D6*1A (CYP2D6.1), CYP2D6*1B (CYP2D6.1), CYP2D6*1C (CYP2D6.1), CYP2D6*1D (CYP2D6.1), CYP2D6*1E (CYP2D6.1), CYP2D6*1XN (CYP2D6.1), CYP2D6*2A (CYP2D6.2), CYP2D6*2B (CYP2D6.2), CYP2D6*2C (CYP2D6.2), CYP2D6*2D (CYP2D6.2), CYP2D6*2E (CYP2D6.2), CYP2D6*2F (CYP2D6.2), CYP2D6*2G (CYP2D6.2), CYP2D6*2H (CYP2D6.2), CYP2D6*2J (CYP2D6.2), CYP2D6*2K (CYP2D6.2), CYP2D6*2L (formerly CYP2D6*41B) (CYP2D6.2), CYP2D6*2M (CYP2D6.2), CYP2D6*2XN (N=2, 3, 4, 5 or 13) (CYP2D6.2), CYP2D6*3A, CYP2D6*3B, CYP2D6*4A, CYP2D6*4B CYP2D6*4C CYP2D6*4D CYP2D6*4E CYP2D6*4F CYP2D6*4G CYP2D6*4H CYP2D6*4J CYP2D6*4K CYP2D6*4L CYP2D6*4X2 CYP2D6*5 CYP2D6*6A CYP2D6*6B CYP2D6*6C CYP2D6*6D CYP2D6*7 (CYP2D6.7), CYP2D6*8 CYP2D6*9 (CYP2D6.9), CYP2D6*10A (CYP2D6.10), CYP2D6*10B (CYP2D6.10), CYP2D6*10C CYP2D6*10D (CYP2D6.10), CYP2D6*10X2 (CYP2D6.10), CYP2D6*11 CYP2D6*12 (CYP2D6.12), CYP2D6*13 CYP2D6*14A (CYP2D6.14A), CYP2D6*14B (CYP2D6.14B), CYP2D6*15 CYP2D6*16 CYP2D6*17 (CYP2D6.17), CYP2D6*18 (CYP2D6.18), CYP2D6*19 CYP2D6*20 CYP2D6*21A CYP2D6*21B CYP2D6*22 (CYP2D6.22), CYP2D6*23 (CYP2D6.23), CYP2D6*24 (CYP2D6.24), CYP2D6*25 (CYP2D6.25), CYP2D6*26 (CYP2D6.26), CYP2D6*27 (CYP2D6.27), CYP2D6*28 (CYP2D6.28), CYP2D6*29 (CYP2D6.29), CYP2D6*30 (CYP2D6.30), CYP2D6*31 (CYP2D6.31), CYP2D6*32 (CYP2D6.32), CYP2D6*33 (CYP2D6.33), CYP2D6*34 (CYP2D6.34), CYP2D6*35 (CYP2D6.35), CYP2D6*35X2 (CYP2D6.35), CYP2D6*36 (CYP2D6.36), CYP2D6*37 (CYP2D6.37), CYP2D6*38 CYP2D6*39 (CYP2D6.39), CYP2D6*40 (CYP2D6.40), CYP2D6*41A (CYP2D6.2), CYP2D6*41B CYP2D6*42 (CYP2D6.42), CYP2D6*43 (CYP2D6.43), CYP2D6*44 (CYP2D6.44), CYP2D6*45A (CYP2D6.45), CYP2D6*45B (CYP2D6.45), CYP2D6*46 (CYP2D6.46), CYP2D6*47 (CYP2D6.47), CYP2D6*48 (CYP2D6.48), CYP2D6*49 (CYP2D6.49), CYP2D6*50 (CYP2D6.50), CYP2D6*51 (CYP2D6.51), CYP2D6*52 (CYP2D6.52), CYP2D6*53 (CYP2D6.53), CYP2D6*54 (CYP2D6.54), CYP2D6*55 (CYP2D6.55), CYP2D6*56 CYP2D6*57 CYP2D6*58 (CYP2D6.58), CYP2E1*1A (CYP2E1.1), CYP2E1*1B (CYP2E1.1), CYP2E1*1C (CYP2E1.1), CYP2E1*1D (CYP2E1.1), CYP2E1*2 (CYP2E1.2), CYP2E1*3 (CYP2E1.3), CYP2E1*4 (CYP2E1.4), CYP2E1*5A (CYP2E1.1), CYP2E1*5B (CYP2E1.1), CYP2E1*6 (CYP2E1.1), CYP2E1*7A (CYP2E1.1), CYP2E1*7B (CYP2E1.1), CYP2E1*7C (CYP2E1.1), CYP2J2*1 (CYP2J2.1), CYP2J2*2 (CYP2J2.2), CYP2J2*3 (CYP2J2.3), CYP2J2*4 (CYP2J2.4), CYP2J2*5 (CYP2J2.5), CYP2J2*6 (CYP2J2.6), CYP2J2*7 (CYP2J2.7), CYP2J2*8 (CYP2J2.8), CYP2J2*9 (CYP2J 2.9), CYP2R1*1 (CYP2R1.1), CYP2R1*2 (CYP2R1.2), CYP2S1*1A (CYP2S1.1), CYP2S1*1B (CYP2S1.1), CYP2S1*1C (CYP2S1.1), CYP2S1*1D (CYP2S1.1), CYP2S1*1E (CYP2S1.1), CYP2S1*1F (CYP2S1.1), CYP2S1*1G (CYP2S1.1), CYP2S1*1H (CYP2S1.1), CYP2S1*2 (CYP2S1.2), CYP2S1*3 (CYP2S1.3), CYP3A4*1A (CYP3A4.1), CYP3A4*1B (CYP3A4.1), CYP3A4*1C (CYP3A4.1), CYP3A4*1D (CYP3A4.1), CYP3A4*1E (CYP3A4.1), CYP3A4*1F (CYP3A4.1), CYP3A4*1G (CYP3A4.1), CYP3A4*1H (CYP3A4.1), CYP3A4*1J (CYP3A4.1), CYP3A4*1K (CYP3A4.1), CYP3A4*1L (CYP3A4.1), CYP3A4*1M (CYP3A4.1), CYP3A4*1N (CYP3A4.1), CYP3A4*1P (CYP3A4.1), CYP3A4*1Q (CYP3A4.1), CYP3A4*1R (CYP3A4.1), CYP3A4*1S (CYP3A4.1), CYP3A4*1T (CYP3A4.1), CYP3A4*2 (CYP3A4.2), CYP3A4*3 (CYP3A4.3), CYP3A4*4 (CYP3A4.4), CYP3A4*5 (CYP3A4.5), CYP3A4*6, CYP3A4*7 (CYP3A4.7), CYP3A4*8 (CYP3A4.8), CYP3A4*9 (CYP3A4.9), CYP3A4*10 (CYP3A4.10), CYP3A4*11 (CYP3A4.11), CYP3A4*12 (CYP3A4.12), CYP3A4*13 (CYP3A4.13), CYP3A4*14 (CYP3A4.14), CYP3A4*15A (CYP3A4.15), CYP3A4*15B (CYP3A4.15), CYP3A4*16A (CYP3A4.16), CYP3A4*16B (CYP3A4.16), CYP3A4*17 (CYP3A4.17), CYP3A4*18A (CYP3A4.18), CYP3A4*18B (CYP3A4.18), CYP3A4*19 (CYP3A4.19), CYP3A5*1A (CYP3A5.1), CYP3A5*1B (CYP3A5.1), CYP3A5*1C (CYP3A5.1), CYP3A5*1D (CYP3A5.1), CYP3A5*1E (CYP3A5.1), CYP3A5*2 (CYP3A5.2), CYP3A5*3A, CYP3A5*3B, CYP3A5*3C, CYP3A5*3D, CYP3A5*3E, CYP3A5*3F, CYP3A5*3G, CYP3A5*3H, CYP3A5*3I, CYP3A5*3J, CYP3A5*4 (CYP3A5.4), CYP3A5*5, CYP3A5*6, CYP3A5*7, CYP3A5*8 (CYP3A5.8), CYP3A5*9 (CYP3A5.9), CYP3A5*10 (CYP3A5.10), CYP3A5*11 (CYP3A5.11), CYP3A7*1A (CYP3A7.1 and CYP3A7.1L), CYP3A7*1B (CYP3A7.1), CYP3A7*1C (CYP3A7.1), CYP3A7*1D (CYP3A7.1), CYP3A7*1E (CYP3A7.1), CYP3A7*2 (CYP3A7.2), CYP3A 7*3 (CYP3A7.3), CYP3A43*1A (CYP3A43.1), CYP3A43*1B (CYP3A43.1), CYP3A43*2A (CYP3A43.2), CYP3A43*2B (CYP3A43.2), CYP3A43*3 (CYP3A43.3), CYP4B1*1 (CYP4B1.1), CYP4B1*2, CYP4B1*3 (CYP4B1.3), CYP4B1*4 (CYP4B1.4), CYP4B1*5 (CYP4B1.5), CYP4B1*6 (CYP4B1.6), CYP4B1*7 (CYP4B1.7), CYP5A1*1A (CYP5A1.1), CYP5A1*1B (CYP5A1.1), CYP5A1*1C (CYP5A1.1), CYP5A1*1D (CYP5A1.1), CYP5A1*2 (CYP5A1.2), CYP5A1*3 (CYP5A1.3), CYP5A1*4 (CYP5A1.4), CYP5A1*5 (CYP5A1.5), CYP5A1*6 (CYP5A1.6), CYP5A1*7 (CYP5A1.7), CYP5A1*8 (CYP5A1.8), CYP5A1*9 (CYP5A1.9), CYP8A1*1A (CYP8A1.1), CYP8A1*1B (CYP8A1.1), CYP8A1*1C (CYP8A1.1), CYP8A1*1D (CYP8A1.1), CYP8A1*1E (CYP8A1.1), CYP8A1*1F (CYP8A1.1), CYP8A1*1H (CYP8A1.1), CYP8A1*1J (CYP8A1.1), CYP8A1*1K (CYP8A1.1), CYP8A1*1L (CYP8A1.1), CYP8A1*2 (CYP8A1.2), CYP8A1*3 (CYP8A1.3), CYP8A1*4 (CYP8A1.4), CYP21A2*8 (89C>T), CYP21A2*9 (655A/C>G), CYP21A2*10 (706delGAGACTAC), CYP21A2*11 (999T>A), CYP21A2*12 (1380T>A), CYP21A2*13 (1383T>A), CYP21A2*14 (1389T>A), CYP21A2*15 (1683G>T), CYP21A2*16 (1762insT), CYP21A2*17 (1994C>T), CYP21A2*18 (2108C>T), CYP21A2*19 (2578C>T), CYP21A2*20A (1380T>A; 1383T>A; 1389T>A), CYP21A2*20B (−126C>T; −113G>A; −110T>C; −103A>G; 89C>T; 655A/C>G; 706delGAGACTAC; exact extension not defined), CYP21A2*20C (−126C>T; −113G>A; −110T>C; −103A>G; 89C>T; 655A/C>G; 706delGAGACTAC; 999T>A; 1380T>A; 1383T>A; 1389T>A; 1683G>T; 1762insT), CYP21A2*20D (−126C>T; −113G>A; −110T>C; −103A>G), CYP21A2*20E (655A/C>G; 1683G>T), CYP21A2*20F (999T>A; 1380T>A; 1383T>A; 1389T>A; 1683G>T; 1762insT), CYP21A2*20G (1380T>A; 1383T>A; 1389T>A; 1683G>T), CYP21A2*20H (1762insT; 1994C>T), CYP21A2*20J (1994C>T; 2108C>T), CYP21A2*20K (−126C>T; −113G>A; −110T>C; −103A>G; 89C>T; 655A/C>G; 706delGAGACTAC), CYP21A2*20L (999T>A; 1380T>A; 1383T>A; 1389T>A; 1683G>T; 1762insT; 1994C>T; 2108C>T), CYP21A2*20M (999T>A; 2578C>T), CYP21A2*20N (655A/C>G; 999T>A), CYP21A2*20P (1683G>T; 1762insT), CYP21A2*20Q, (1683G>T; 1994C>T; 2108C>T), CYP21A2*20R (−126C>T; −113G>A; −110T>C; −103A>G; 89C>T), CYP21A2*20S (−126C>T; −113G>A; −110T>C; −103A>G; 89C>T; 655A/C>G), CYP21A2*20T (655A/C>G; 1380T>A; 1383T>A; 1389T>A; 1683G>T; 1994C>T), CYP21A2*20U (1683G>T; 1762insT; 1994C>T; 2108C>T), CYP21A2*20V (1380T>A; 1383T>A; 1389T>A; 1683G>T; 1762insT; 1994C>T; 2108C>T), CYP21A2*9+CYP21A2*17 (655A/C>G+1994C>T), CYP21A2*21 (1203G>C), CYP21A2*22 (692C>T; 2578C>T), CYP21A2*23 (1713G>A), CYP21A2*24 (2058G>A; 2578C>T), CYP21A2*25 (2668delGGinsC), CYP21A2*26 (1779G>C), CYP21A2*27 (2339G>A), CYP21A2*28 (2669G>C), CYP21A2*29 (2063C>T), CYP21A2*30 (66G>A), CYP21A2*31 (295A>G), CYP21A2*32 (1748G>A), CYP21A2*33 (2109G>C), CYP21A2*34 (2109G>A), CYP21A2*35 (2265G>C), CYP21A2*36 (669C>A), CYP21A2*37 (989delTGinsA), CYP21A2*38 (1158delAGG), CYP21A2*39 (387G>A), CYP21A2*40 (1988C>T), CYP21A2*41 (2029delTCCAGCTCCC), CYP21A2*42 (2316duplCCTGGATGAGACGGTC), CYP21A2*43 (1780T>G), CYP21A2*44 (2647delT), CYP21A2*45 (89C>A), CYP21A2*46 (141delT), CYP21A2*47 (191G>A), CYP21A2*48 (1626C>T), CYP21A2*49 (2127C>T), CYP21A2*50 (317A>T), CYP21A2*51 (366G>T), CYP21A2*52 (1016G>C), CYP21A2*53 (1713G>T), CYP21A2*54 (2103G>A), CYP21A2*55 (2494G>A), CYP21A2*56 (655A/C>G; 2494G>A), CYP21A2*57 (1684T>G), CYP21A2*58 (1740C>T), CYP21A2*59 (2102C>T), CYP21A2*60 (996T>A), CYP21A2*61 (2665C>T), CYP21A2*62 (82insC), CYP21A2*63 (Dupl62-172), CYP21A2*64 (1624T>C), CYP21A2*65 (1981C>A), CYP21A2*66 (2130T>G), CYP21A2*67 (2524C>T), CYP21A2*68 (666A>G), CYP21A2*69 (992insA), CYP21A2*70 (1579delA), CYP21A2*71 (1752G>A), CYP21A2*72 (2248G>A), CYP21A2*73 (2453C>T), CYP21A2*74 (−126C>T; −113G>A; −110T>C; −103A>G; 43G>A; 89C>T), CYP21A2*75 (89C>T; 185A>T), CYP21A2*76 (1121delC), CYP21A2*77 (1713G>C), CYP21A2*78 (1744C>A), CYP21A2*79 (2064G>C), CYP21A2*80 (2253C>A), CYP21A2*82 (2498G>A), CYP21A2*83 (3G>A), CYP21A2*84 (749G>A), CYP21A2*85 (56G>A), CYP21A2*86 (2668insC), CYP21A2*87 (2668C>T), CYP21A2*88 (1208insT), CYP21A2*89 (64insT), CYP21A2*90 (1355C>T), CYP21A2*91 (185A>T), CYP21A2*92 (1689A>C), CYP21A2*93, CYP21A2*94 (327T>C), CYP21A2*95 (2522C>T), CYP21A2*96 (2093G>A), CYP21A2*97 (126delC), CYP21A2*98 (992insA), CYP21A2*99 (2135C>T), CYP21A2*100 (2657G>T), and CYP21A2*101 (1762delT).

B. Efficacy Markers

In addition to tests for drug metabolism enzymes (DMEs), much is also known about markers that predict if a drug will be effective for a select number of therapeutic areas. For example, one can look for mutations such as B₂AR (β-adrenergic receptors-important for patients being treated for asthma with Albuterol), ERCC1 (excision repair cross complementing gene 1; may affect DNA repair capabilities), ERCC2 (excision repair cross complementing gene 2; may affect DNA repair capabilities), MDR1 (multidrug resistance gene 1; determines drug absorption in tumor cells), XRCC1 (X-ray repair cross complementing gene 1), VKORC1 (an enzyme involved in vitamin K recycling and the drug target for warfarin) and 5HTT (5-hydroxytryptamine transporter; a.k.a. SLC6A4-determines effectiveness of drugs used to treat depression and other CNS disorders. DMEs that predict drug toxicity could also be considered efficacy markers since the presence of adverse effects may limit therapeutic efficacy and may require discontinuation of an otherwise effective treatment. Irinotecan (Camptostar) has been approved for the standard therapy of colorectal cancer. Although irinotecan is a promising chemotherapeutic agent, the most common unwanted side effects are bone marrow toxicity leading to abnormal blood counts, in particular leucopenia and ileocolitis. Irinotecan is metabolized to form active SN-38, which is further conjugated and detoxified by UDP-glucuronosyltransferase (UGT) 1A1 enzyme. Genetic polymorphisms of the UGT1A1 would affect an interindividual variation of the toxicity by irinotecan via the alternation of bioavailability of SN-38. Determination of the UGT1A1 genotypes can be clinically useful for predicting severe toxicity by irinotecan in cancer patients.

C. Identifying Patients with Genetic Disorders

Patients suffering from various genetic disorders can be identified using genomic screening techniques. To ensure that the genomic screening techniques are being performed correctly, reference controls that contain genetic mutations associated with such disorders can be prepared using the techniques described herein.

One example of a disorder that can be identified through genomic screening is Cystic Fibrosis. More than 1000 mutations of the CFTR gene are listed in the Cystic Fibrosis Mutation Data Base. Mutations that can be present in suitable reference controls include 1898+IG>A, 1148T, 2184delA, 1078delT, 394delTT, S1235R, and combinations of the IVSB polyT tract variant alleles 5T, 7T, and 9T. Although S1235R is not currently included in a screening panel, this mutation may be associated with disease, so reference controls including this mutation can potentially be important.

Another mutation that can be confirmed via genomic screening is a MTHFR deficiency. MTHFR (Methylenetetrahydrofolate reductase) is important in folate metabolism, and mutations in this gene may lead to increased concentrations of homocysteine. Increased concentrations of homocysteine are associated with severe neurologic impairment. One thermolabile variant, 677C>T, does not appear to be associated with neurologic symptoms, but may be associated with an increased risk for vascular disease, and has an estimated frequency of up to 24% depending on the population. Clinical testing for MTHFR 677C>T is widespread as part of a cardiovascular risk panel.

Another type of mutation that can be identified is present in the HFE (hemochromatosis) gene, with a frequency believed to be in excess of 10% in the Caucasian population. These mutations are associated with hereditary hemochromatosis. Reference controls with mutations in C282Y, C282Y/H63D, H63D, and S65C, with heterozygotes such as H63D/S65C, can be developed. The proximity of S65C to H63D can lead to incorrect identification of the genotype, so reference controls with both mutations can be important controls for validating clinical assays.

Other mutations are associated with a risk for thombosis. These genes are typically associated with coagulation factor V (F5) and prothombin (F2), and mutations such as FVL and the prothrombin polymorphism 20210G>A.

Huntington's disease is a severe neurologic disorder resulting from an increased number of CAG repeats in the Huntington's (HD) gene. Individuals carrying alleles with repeats in a certain range do not display symptoms of Huntington disease, but run the risk of transmitting the disorder to their children. The repeat number can be measured for purposes of genetic counseling.

Fragile X syndrome is the most common inherited form of mental retardation. It is caused by expansion of the CGG-repeat region and abnormalities in the methylation pattern of the FMR1 gene. The normal repeat number is from 6 to ˜55. Repeats in the range of ˜55 to 200-230 are considered pre-mutation or intermediate range, and repeats in excess of 200-230 are in the full mutation, disease-causing, range. Although individuals with repeats in the intermediate range may be asymptomatic, they are at risk of transmitting a full-expansion mutation allele to their offspring. As with Huntingon's disease, use of reference controls with a fixed number of repeats can be used to confirm the accuracy of the count of these repeats.

Defects in the connexin 26 gene (GJB2) are thought to be responsible for ˜50% of all nonsyndromic autosomal recessive deafness, and ˜70% of the currently identified connexin 26 mutations are of the type carried by DUK19946. Since the link between connexin 26 and deafness was established in the 1990s, a demand for clinical testing for mutations in the connexin 26 gene has developed. Reference controls for mutations in this gene can represent an important positive control.

Deletions in the α-globin gene cluster are common in certain populations and cause α-thalassemia with various degrees of severity, depending on the type of deletion. Several different deletions have been identified in the α-globin gene cluster, which deletions can form the basis for suitable reference controls. Examples include type 1 deletions (both α-globin genes deleted; 1 heterozygous SEA deletion and 1 heterozygous FIL deletion), and type 2 deletions (1 gene deleted; heterozygous).

There are also various known point mutations in the β-hemoglobin (HBB) gene: the Hb S mutation, which is responsible for sickle cell disease, and the Hb C mutation, which is associated with chronic hemolytic anemia. The Hb S and Hb C mutations occur in the same codon. Hb S leads to the substitution of valine for glutamic acid, whereas Hb C leads to the substitution of lysine. The presence of both mutations in a compound heterozygous state cause Hb SC disease, which has characteristics of both sickle cell and Hb C disease. Because the 2 mutations occur in such close proximity to one another, they are often tested for simultaneously. A reference control for both mutations can be used for genomic assays.

### II. Methods for Pre-Screening Samples

Using the methods described herein, biological samples are pre-screened to ensure that they have the mutation of interest. Before pre-screening the samples, one should determine the types of mutations for which one is screening. Once this is determined, patient populations can be pre-screened, based on a variety of factors, to minimize the sample size needed to identify individuals that include the mutation. After performing initial genomic screening on the samples to identify one or more samples which include the mutation of interest, the patients with these mutations can optionally be recalled to obtain additional biological material. This material can optionally be thoroughly sequenced to confirm the presence of the mutation of interest. The biological material can be immortalized, so it can provide a steady, on-demand source of the reference controls, or, alternatively, the cells themselves can be the reference controls.

The genomic DNA can then be screened against a plurality of assays to confirm that the DNA is suitable for use as a reference control in a variety of assay methods. The initial test validates the reference controls. Once validated, the reference controls can be used to develop additional assay methods, particularly primers for use in these methods, and can also be used to ensure that the laboratories are correctly performing the assays.

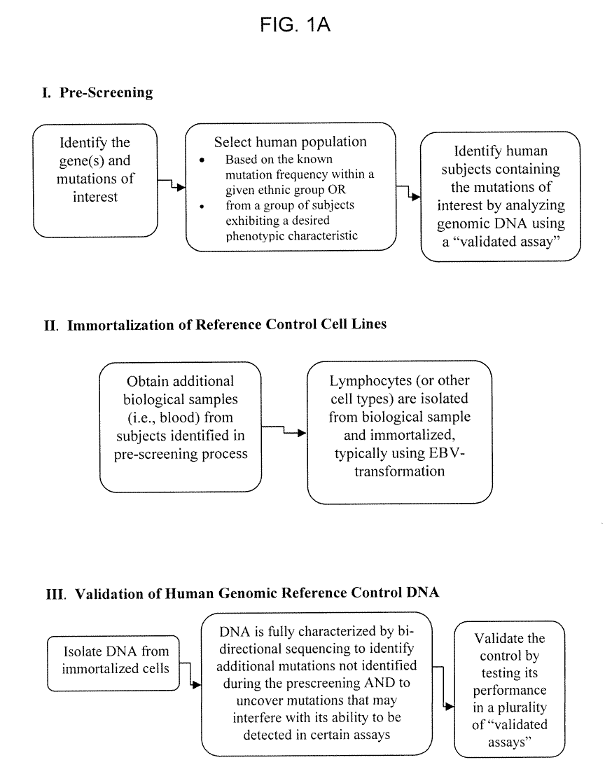

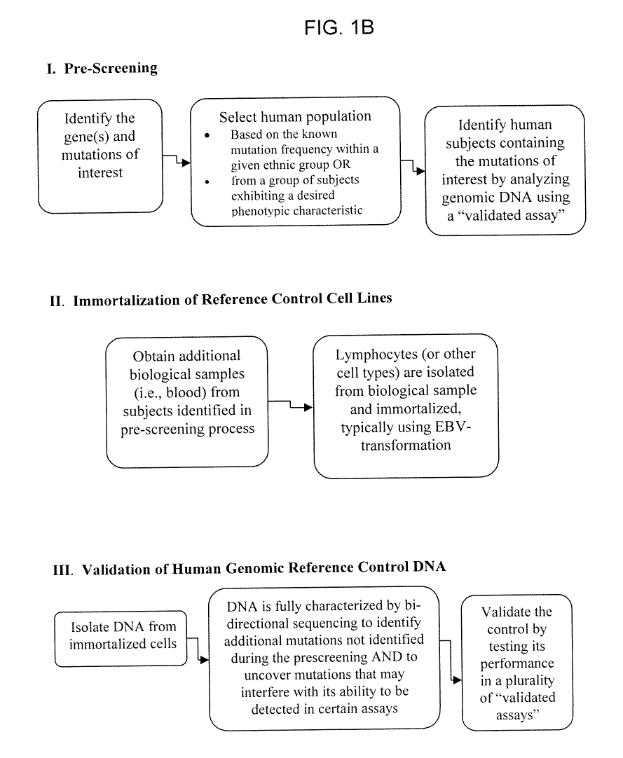

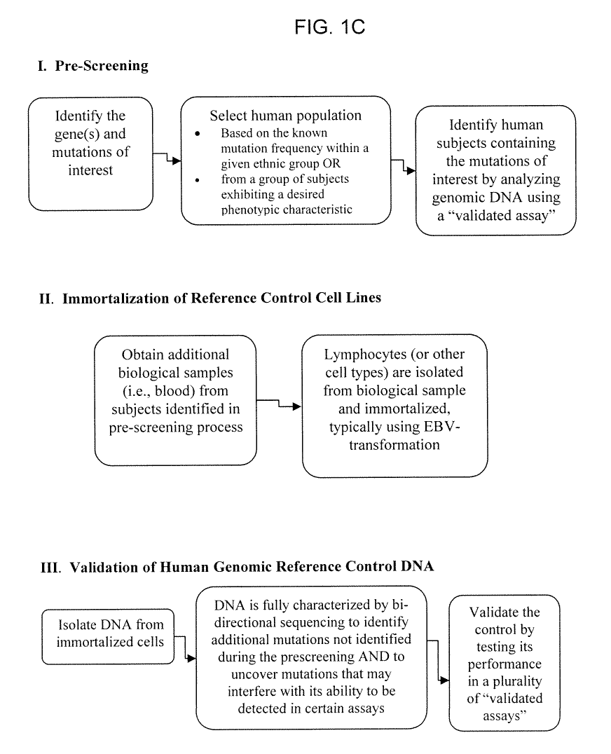

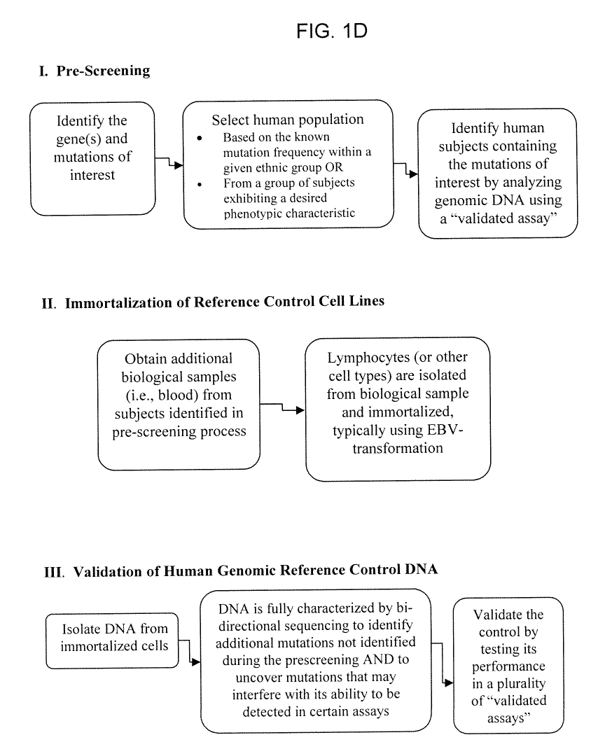

FIG. 1 is a schematic illustration of one embodiment of the methods described herein. As shown in the Figure, pre-screening involves identifying the genes and mutations of interest, selecting human populations that have these mutations, and identifying human subjects containing the mutations by using a validated assay to analyze their genomic DNA. Immortalization typically involves obtaining additional samples from subjects identified in the pre-screening process, and then using EBV or other immortalization methods. Validation of the reference controls is done by isolating DNA from the immortalized cells, optionally fully characterizing the DNA, and validating the control by testing its performance in a plurality of validated assays.

These individual steps are described in more detail below.

A. Identification of Mutations of Interest

The first step in preparing appropriate reference controls is identifying the types of mutations for which one is interested in screening. Particularly when the reference controls are to be used in clinical trials and/or patient care, these mutations will be in genes associated with drug metabolism or response to a particular therapeutic agent, rather than mutations associated with the presence of a genetic disorder. Once one determines which mutations are to be identified, one can identify one or more human subjects with genomic DNA that includes the mutations of interest. This genomic DNA can serve as a reference control, provided it becomes a “validated” reference control (one in which the mutation(s) were correctly detected using one or more assays designed to detect those specific mutation(s)).

B. Identification of the Percentage of the Population that has the Mutation of Interest

When obtaining human genomic DNA from human subjects, it can be advantageous to determine, where possible, the percentage of the population with DNA containing the mutation of interest. If this percentage is known, then a suitable population can be selected such that, statistically, one or more subjects will have the mutation of interest.

In those instances where particular ethnic groups have a higher preponderance of the mutation of interest, a pre-screening step can be performed to identify samples from that particular ethnic group.

C. Phenotypic Pre-Screening

In those instances where the subjects containing the mutation can be identified phenotypically (e.g., by the presence of an illness or other readily identifiable feature), a pre-screening step can be performed to identify samples from that particular patient population.

D. Obtaining Consent

It is often desirable to pre-screen properly consented normal human volunteers, and examine the genotype or genetic profile of the volunteers. Then, those volunteers with genotypes of interest can be recalled in order to obtain a sufficient amount of lymphocytes or other suitable cell types for preparation of immortalized cells or storage until needed.

To ensure that the volunteers have given consent, one can require that a proper consent document is obtained. One can use a tiered approach, allowing the sponsor to screen first but retain enough of the patient identifiers in order to recall the patient. A de-identification system can be used that allows the clinic where patients come for donating samples to send samples with a simple unique random barcode number. This way, the actual patient can be anonymous to the lab while it is still possible to recall patients having appropriate genomic sequences. Alternatively, the patient identifiers can be left on the samples during the pre-screening step, and the de-identification system will be used when a group of subjects donate an additional biological sample for the final immortalization step.

It is also advantageous to have a cooperative clinical partner who has access not only to sufficient patient population numbers, but also to an ethnically diverse population. As mentioned previously, many SNPs and variants are common to particular ethnic groups (e.g., CYP2D6*7 appears mostly in patients of Hispanic origin). The clinic partner can advantageously focus on recruiting a particular ethnic volunteer population when the genomic sequence and corresponding mutation is found only in a particular race. Clinical partners can have this information, and also may have already obtained informed consent.

E. Types of Biological Samples from which to Isolate the Reference Control

The source of the DNA is from a human, so human genomic DNA is used as the reference control material in this instance. The DNA can be present in any nucleic acid-containing sample of tissues, bodily fluids (for example, blood, serum, plasma, saliva, urine, tears, semen, vaginal secretions, lymph fluid, cerebrospinal fluid or mucosa secretions), individual cells or extracts of the such sources that contain the nucleic acid of the same, and subcellular structures such as mitochondria or chloroplasts, using protocols well established within the art.

In a preferred embodiment, the nucleic acid has been obtained from a human to be pre-screened for the presence of one or more genetic sequences that can be diagnostic for, or predispose the subject to, a medical condition or disease. As an alternative to prescreening patient populations, one can screen cell cultures commonly available for the genetic sequence of interest, and then expand and preserve the cell clones for future continuous supply.

F. Analyzing Samples for Mutations of Interest

Human subjects with genomic DNA that includes the mutations of interest are typically identified using a previously validated assay. The term “validated assay” means an assay that has been shown able to detect previously sequenced samples that are known to possess the mutation that the assay was designed to detect. As discussed in more detail below, the detection is primarily done by hybridizing the genomic DNA in the sample with a primer. The primer is designed, typically using known genomic sequence information, to hybridize to a region that includes the mutation of interest (a “hybridization region”).

Genomic screening methods, for example, pharmacogenomic screening methods, are typically performed by taking a patient sample, isolating DNA from the sample, and subjecting the DNA to hybridization conditions in the presence of primers which include the complement of the SNP of interest, where the primers bind to samples including genomic DNA that possesses the SNP of interest. This primer binding is often used to generate an amplicon via a process such as polymerase chain reaction (PCR). Then, the presence of a mutation of interest is confirmed, for example, using a fluorescent tag on a primer or on a probe.

G. Detection of False Positive Results or Pseudogenes

It can also be important to be able to obtain reference controls which can be used to identify either false positive results in a particular method (e.g., amplification of a pseudogene or primers annealing to other sequences in the human genome caused by common SNPs not known by others where a particular diagnostic product does not allow for discrimination of these sequences). If in the test, the result is positive for a mutation, yet the validated reference control is known not to contain the mutation, the reference control then has detected a false positive which may be attributable to amplification of pseudogene.

H. Detection of Samples with Multiple Mutations of Interest

Once samples with a desired mutation are identified, additional steps can be performed to pre-screen the samples for the ability of the human genomic DNA within the cells to function as reference controls. One such step involves the selection of samples which contain two or more mutations of interest.

Certain genetic mutations result in a patient/volunteer which does not metabolize drugs at the same rate as a normal patient/volunteer, whereas other mutations result in a patient which will not benefit from a particular drug therapy. When a single sample includes two or more mutations, these can be two or more mutations of a gene that controls metabolism, or which is responsible for a patient/volunteer not responding to a particular therapy, or combinations thereof.

In some embodiments, one can identify patients/volunteers who will not respond normally to a given therapy, due to variations in their ability to metabolize drugs, generally, or their non-responsiveness to a given therapy. By preparing one or more reference controls which can verify that the screening method identifies both types of patients/volunteers, clinical trial data can be more accurate, and patient prescribing can be done with a higher degree of effectiveness.

In a preferred embodiment, the patients/volunteers have genes containing two or more known mutations, so that the reference controls are effective for screening for more than one mutation. Of course, mixtures of reference controls can be prepared which include a plurality of mutations of interest, so that one platform can be used to show positive results for all mutations of interest.

### III. Immortalization of Cells

In order to have a constant supply of DNA belonging to specific genotypes, such as the reference controls described herein, it is advantageous to immortalize cells including one or more mutations of interest. Although other methods for immortalizing cells are known, one way to immortalize cells involves EBV-immortalization. This is an effective procedure for inducing long-term growth of human B-lymphocytes, and the resulting EBV-transformed cells can serve as reliable sources of genomic DNA. This can be particularly advantageous in the context of performing clinical trials.

EBV-transformation of peripheral blood lymphocytes is a relatively standard laboratory procedure. It typically takes around six to eight weeks to generate large amounts of EBV-transformed cells, and these cells can be indefinitely grown as a continuing source of genetic material.

The lymphocytes are obtained from whole blood samples (typically in 12-15 ml volumes), typically collected from donors in a sterile citrate or heparin tube. The blood samples are typically stored at room temperature, and ideally are shipped to an appropriate facility for conducting the EBV transformation within 24 hours. Ideally, one will obtain a properly executed donor consent form, approved by an institutional review committee, before the genomic DNA is used for research. Other tissue types such as kidney, liver, and lung can also be immortalized using similar processes as those described below.

A typical protocol for EBV-transformation of peripheral blood lymphocytes obtaining appropriate peripheral blood lymphocytes from one or more donors. The lymphocytes are cultured in an appropriate tissue culture medium (such as RPMI 1640 supplemented with 10% fetal bovine serum and essential amino acids). The culture is then infected with EBV supernatant.

The infected cells typically start showing morphological changes after three to four days, at which point dividing cells can be seen as dumbbell shaped structures under an inverted microscope. There are typical morphological changes manifested by an actively growing cell culture that are visible to the naked eye. One example of this is the cellular clumps that tend to form six to eight weeks from infection.

IV. Isolation of Reference Controls from Normal and/or Immortalized Cells

Although in one embodiment, the reference controls can be the actual cell lines that include the mutations of interest, the reference controls described herein are ideally the genomic DNA that comprises the entire genome present in these cells. Once the cell lines are screened, isolated, and optionally immortalized, the reference controls are obtained by extracting the DNA from the cells.

There are numerous known DNA extraction methods, including solution-based methods, magnetic bead-based methods, spin-column based methods and phenol-chloroform based protocols, and those of skill in the art can readily select an appropriate technique for isolating genomic DNA from the cells described above. Ideally, the DNA extraction protocol avoids using toxic compounds, and is fast and reliable.

One example of a suitable type of purification kit is PUREGENE™ DNA Purification Kit (Gentra Systems). It can be used to purify genomic, mitochondrial, and viral DNA. The PUREGENE system works via alcohol and salt precipitation. Using this kit, cells are lysed with an anionic detergent in the presence of a DNA stabilizer that inhibits DNase activity. RNA and proteins are then digested, and other contaminants removed by salt precipitation. The DNA is then alcohol precipitated and dissolved in a DNA stabilizer. This sequence of steps is relatively rapid, taking approximately 2 hours, is very consistent, and produces high yields of DNA comparable to spin-column based methods and phenol-chloroform based protocols.

### V. Methods for Pre-Screening Putative Reference Controls

Not only is it important for a particular sample of genomic DNA to have a mutation of interest, it also must be possible to detect the mutation of interest. Thus, it is advantageous to pre-screen putative reference controls for their ability to be detected in a plurality of genomic screening assays. When the biological samples are initially screened for the presence of the mutations of interest, they are subjected to a validated assay, and optionally are analyzed more thoroughly using DNA sequencing to confirm the presence of the mutation. This confirms that the mutation(s) present in the reference control can be detected by the validated assay, but does not confirm that the mutation can be detected by any assay method.

The samples used to validate the assay may not have included all possible genetic variations in the “hybridization region” (also known as a “loci of interest”). Accordingly, even if the assay would detect the vast majority of samples that include the mutation of interest, it is possible that some samples that also include the same mutation of interest will not be detected by the assay. For example, in the PCR-based assay described above, binding of the PCR primers or diagnostic probe to the hybridization region in a particular sample may be compromised due to the presence of other polymorphisms that interfere with proper annealing.

Biological samples from the patients are evaluated in these assays to determine that the sample contains the mutation. The presence of the mutation can be, but need not be, confirmed by a more thorough DNA sequencing process, which can be, but which need not be, performed in a series of contiguous sequencing processes to confirm the results and to determine the presence or absence of other mutations within the reference control.

The use of a previously validated assay will identify some, and perhaps, most samples that include a mutation of interest, but will not necessarily identify all samples that include the mutation of interest. For example, there may be one or more additional mutations in the sample near the region of the mutation of interest, where hybridization with the primer or diagnostic probe in the validated assay would otherwise occur. The presence of the additional mutation(s) may inhibit the binding of the DNA in the sample to the “validated” primer, so the assay may not detect all samples that include the mutation. That said, many assays are in commercial use and are considered validated assays because they are capable of detecting the mutation of interest in the vast majority of samples that are tested, and the primer and/or probe, for example, are based on the known probabilities of there being other mutations in the samples in the hybridization region that would otherwise inhibit primer binding. Because a particular probe, even one used in a validated assay designed for detecting a particular mutation of interest, may not be capable of identifying every sample that has the mutation, the corollary is also true—a particular reference control may not work with all assays. For this reason, it can be advantageous to pre-screen (and thus validate) the reference control against a plurality of screening assays.

A plurality of screening methods are advantageously used to validate the reference control, that is, to confirm that the reference control can be used to validate the accuracy of a plurality of assays when they are run in a lab, or to validate a primer that is being developed for use in an assay (and, hence, validate the assay).

### VI. Various Genomic Assay Methods

There are a number of known genomic assay methods for which the reference control can be used in testing. As discussed above, most involve hybridizing a primer with a DNA sample that may or may not include a SNP of interest. A diagnostic primer and/or probe can be tagged to permit rapid identification. Once hybridization has occurred, the DNA can be amplified, and the tagged primer and/or probe is detected. As discussed above, validated primers can be used to confirm the validity of reference controls. Once the reference controls are validated, they can be used in commercially available assays as a reference control, and can be used to validate primers that are designed for use in these or other assays to determine the presence or absence of a particular mutation. Representative commercially available assays that can be validated, or for which primers can be developed, using the reference controls, are described below.

Representative examples of assay platforms include, but are not limited to, SNP analysis using the Sequenom™ MassARRAY, Sequenom™ iPLEX, Sequenom™ hME, Illumina Bead stations, fragment analysis using gel or capillary electrophoresis, the ABI Taqman™ Allelic Discrimination assay, Roche's Amplichip technology, Third Wave's Invader assay, pyrosequencing methods, MGB Eclipse™; and TM Biosciences genotyping assays. These and other commercially available genotyping assay platforms can be used to detect the presence or absence of a polymorphism, such as a SNP. Thus, while exemplary assay methods are described herein, the invention is not so limited.

In one embodiment, genotyping is performed using genomic DNA obtained from any tissue or fluid sample of a subject that provides genomic DNA. For example, the presence or absence of one or more polymorphic allele in a subject's nucleic acid can be detected simply by starting with any sample comprising a nucleated cell. DNA for genotyping can be isolated from a variety of sources including a whole blood sample by procedures well known to those of skill in the art. These procedures can be conducted using a variety of commercially available kits such as, for example, the Puregene™ DNA Isolation Kit (Gentra Systems, Inc., Minneapolis, Minn.). DNA Isolation Kit for Blood (Roche Diagnostics Corporation), GenomicPrep™ Blood DNA Isolation Kit (Amersham Biosciences Corp., Piscataway, N.J.), PAXgene Blood DNA Kit (QIAGEN Inc., Valencia, Calif.), GNOME™ Whole Blood DNA Isolation Kit (Qbiogene, Inc., Carlsbad, Calif.) and Wizard™ Genomic DNA Purification Kit (Promega U.S., Madison, Wis.).

Generally, assay methods known in the art for determining a genotype can be based on procedures that involve, without limitation, polymerase chain reaction (PCR)-based analysis, sequence analysis, and electrophoretic analysis. One commercially available non-limiting example of a PCR-based analysis includes a Taqman™ allelic discrimination assay available from Applied Biosystems. Non-limiting examples of sequence analysis include Maxam-Gilbert sequencing, Sanger sequencing, capillary array DNA sequencing, thermal cycle sequencing, solid-phase sequencing, sequencing with mass spectrometry such as matrix-assisted laser desorption/ionization time-of-flight mass spectrometry, and sequencing by hybridization. Non-limiting examples of electrophoretic analysis include slab gel electrophoresis such as agarose or polyacrylamide gel electrophoresis, capillary electrophoresis, and denaturing gradient gel electrophoresis.

Other non-limiting examples of methods for genotyping at a polymorphic site include the INVADER™ assay from Third Wave Technologies, Inc., restriction fragment length polymorphism (RFLP) analysis, allele-specific oligonucleotide hybridization, a heteroduplex mobility assay, and single strand conformational polymorphism (SSCP) analysis.

In one embodiment, the nucleic acid region containing the SNP can be amplified, for example, by PCR techniques using a sense and an anti-sense primer and a detection probe (e.g., TaqMan™ platform). Non-limiting examples of amplification techniques include PCR (U.S. Pat. Nos. 4,698,195; 4,800,159; 4,683,195; 4,683,202), ligase chain reaction (“LCR”) (Landegran et al. “A Ligase-Mediated Gene Detection Technique.” Science 241: 4869 (Aug. 26, 1988) pp. 1077-80, and Nakazawa et al. PNAS 91: 360, 1994), self-sustained sequence replication (Gutaelli, J. C. et al., PNAS 87: 1874, 1990), isothermal nucleic acid sequence based amplification (NASBA), transcriptional amplification system, Q-Beta replicase, or any other nucleic acid amplification method, followed by the detection of the amplified molecules using techniques well known to those of skill in the art. In this embodiment, a nucleic acid probe can be annealed to the locus of interest in a sample in an allele-specific manner during thermocycling. The probe, which comprises a fluorescent dye and a quencher, is annealed between the forward and reverse primer sites. As the DNA polymerase extends the primers, it also degrades the annealed probe, allowing the dye to be removed from under the influence of the quencher, thereby becoming detectable. Since an increase in fluorescence signal occurs only if the amplified target sequence is complementary to the probe, the fluorescence signal generated by PCR amplification indicates which alleles are present in the sample. An example of a commercially available PCR-based platform using a fluorescently-labeled probe is Taqman™. Other fluorescently-labeled probe platforms are known in the art.

Methods for preparing PCR primers and probes are well known in the art, and are described, for example, in Sambrook et al. (1989), Ausubel et al. (1994) and Innis et al. (1990). PCR primer pairs can be derived from known sequences, for example, by using computer programs intended for that purpose such as Primer (Version 0.5, 1991, Whitehead Institute for Biomedical Research, Cambridge Mass.). For example, OLIGO™ version 6 software (Molecular Biology Insights, Inc., Cascade, Colo., www.oligo.net) is useful for the selection of PCR primer pairs of up to 100 nucleotides each. Similar primer selection programs have incorporated additional features for expanded capabilities and include PrimOU primer selection program (Genome Center at University of Texas, South West Medical Center, Dallas Tex.), Primer3, version 0.9, primer selection program (Whitehead Institute/MIT Center for Genome Research, Cambridge Mass.), PrimeGen program (UK Human Genome Mapping Project Resource Centre, Cambridge UK) and other oligonucleotide selection methods known to those of skill in the art.

The sequences complementary to the primer pairs can be separated by as many nucleotides as the PCR technique will allow. However, one of skill in the art knows that there are practical limitations of subsequent assaying procedures, which may dictate the number of nucleotides between the sequences complementary to the primer pairs. Further, the length of the sequence used for probes can be minimized using a minor groove binder (MGB) linked to the probe sequence in order to discriminate between two possible nucleotides at the SNP site. MGBs are known to those of skill in the art (U.S. Pat. Nos. 5,801,155; 6,084,102; 6,426,408; 6,312,894; 6,683,173).

Amplified nucleic acids can be assayed to determine the genotype by any of a variety of methods, including but not limited to allele-specific oligonucleotide (ASO) probing, differential restriction endonuclease digestion, ligase-mediated gene detection (LMGD), gel electrophoresis, oligonucleotide ligation assay (OLA), exonuclease-resistant nucleotides, and genetic bit analysis (GBA). Additional methods of analysis can also be useful, such as fluorescence resonance energy transfer (FRET) (Wolf et. al., PNAS 85: 8790-94, 1988). SSCP (single strand confirmation polymorphism) and DHPLC (denaturing high-performance liquid chromatography) can also be employed. Any of these or other known methods can be employed to determine the presence or absence of polymorphic alleles. The methods employed or compositions used are not intended to be limited to any one polymorphism and should be construed to encompass all polymorphisms described herein.

In one embodiment, ASO probes are used in assays for determining the presence or absence of polymorphic alleles. Accordingly, there is provided a method comprising assaying nucleic acid for the presence or absence of one or more polymorphic alleles by contacting the nucleic acid with an ASO probe(s) under conditions suitable to cause the probe to hybridize with nucleic acid encoding the polymorphic allele, but not with nucleic acid encoding the non-polymorphic allele, and detecting the presence or absence of hybridization.

Antisense oligonucleotides can be prepared as polynucleotides complementary to nucleotide sequences comprising a DNA that contains the polymorphic allele. For example, oligonucleotide probes are synthesized that will hybridize, under appropriate annealing conditions, exclusively to a particular amplified nucleic acid sequence that contains a nucleotide(s) that distinguishes one allele from other alleles. The probes can be discernibly labeled so that when the polymorphic allele-specific oligonucleotide probe hybridizes to the sequence containing the polymorphic allele, it can be detected, and the specific allele is thus identified.

In another embodiment, several probes capable of hybridizing specifically to allelic variants, such as SNPs, are attached to a solid phase support, e.g., a gene chip. Oligonucleotides can be bound to a solid support by a variety of processes, including lithography. Mutation detection analysis using these chips comprising oligonucleotides is known in the art (Cronin et al., Human Mutation 7: 244, 1996). A gene chip can comprise all the allelic variants of at least one polymorphic region of a gene. The solid phase support is then contacted with a test nucleic acid and hybridization to the specific probes is detected. Accordingly, the identity of numerous allelic variants of one or more genes can be identified in a simple hybridization experiment.

In another embodiment of the invention, either of the genomic DNA's amplified nucleic acid product or the ASO probes can be bound onto two solid matrixes (e.g., nylon, nitrocellulose membrane, and the like) by standard techniques and then each membrane can be placed into separate hybridization reactions with an ASO probe or the amplified nucleic acid, respectively. For example, if the amplified nucleic acid were bound onto a solid matrix, one hybridization reaction would utilize an oligonucleotide probe specific for the polymorphic allele under conditions optimal for hybridization of this probe to its complement. The other hybridization reaction would utilize an oligonucleotide specific to the polymorphic allele under conditions optimal for hybridization of that probe to its complement. Accordingly, the ASO probes may bear the same label, but will still be distinguishable because they are hybridized in separate chambers.

This technique permits the determination of whether the genomic DNA comprises the polymorphic allele and also whether the genomic DNA is a heterozygote or a homozygote. If an ASO probe is found to bind to genomic DNA's nucleic acid on only one membrane, then the genomic DNA is homozygous for that particular allele which the ASO probe was designed to bind. If the ASO probes are found to hybridize the genomic DNA's nucleic acid on both membranes, then the genomic DNA is heterozygous.

The ASO probes of the present invention can be about 7 to about 35 nucleotides in length, preferably about 15 to 20 nucleotides in length, and are complementary to a nucleic acid sequence encoding at least the polymorphic nucleotide. Those of skill in the art will understand that other ASO probes may be designed using the sequence information provided herein. For probe design, hybridization techniques and stringency conditions, see, for example, Ausubel et al., (eds.) Current Protocols In Molecular Biology, Wiley Intersciences, N.Y., sections 6.3 and 6.4 (1987, 1989).

The ASO probes may be discernibly “labeled.” As used herein, the term “label” in its various grammatical forms refers to atoms and molecules that are either directly or indirectly involved in the production of a detectable signal to indicate the presence of a complex (e.g., radioisotope, enzyme, chromogenic or fluorogenic substance, a chemiluminescent marker, or the like). Any label can be linked to or incorporated in a probe such as an ASO probe. These atoms or molecules can be used alone or in conjunction with additional reagents. Such labels are themselves well known in clinical diagnostic chemistry.

One of skill in the art can readily determine such conditions for hybridization based upon the nature of the probe used, factoring into consideration, time temperature, pH, and the like.

In other embodiments, assaying for the presence or absence of polymorphic alleles comprises cleaving the amplified nucleic acid with a restriction endonuclease, wherein the restriction endonuclease differentially cleaves nucleic acid encoding a polymorphic allele as compared to the wild type. If a particular amplified nucleic acid contains a sequence variation associated with an allele of a polymorphism, and this sequence variation is recognized by a restriction endonuclease, then the cleavage by the enzyme of a particular nucleic acid sequence can be used to confirm the presence of the allele. In this process, amplified nucleic acid is digested and the resulting fragments are analyzed by size or movement through a gel. The presence or absence of nucleotide fragments, corresponding to the endonuclease cleaved fragments, determines which allele is present. A restriction endonuclease suitable for use in the practice of the present invention can be readily identified by one of skill in the art.

In other embodiments, assaying for the presence or absence of polymorphic alleles comprises hybridizing the amplified nucleic acid with a pair of oligonucleotide probes to produce a construct, wherein a first probe of the pair is labeled with a first label and a second probe of the pair is labeled with a second label, such that the first label is distinguishable from the second label, and the probes hybridize adjacent to each other. This is followed by reacting the construct with a ligase in a reaction medium, and then analyzing the reaction medium to detect the presence or absence of a ligation product comprising the first probe and the second probe.

In an LMGD-type assay, a pair of oligonucleotide probes can be synthesized that will hybridize adjacently to each other at the specific nucleotide that distinguishes the polymorphic alleles from the wild types (U.S. Pat. No. 6,008,335). Each of the pair of specific probes is labeled in a different manner, and when hybridized to the allele-distinguishing DNA segment, both probes can be ligated together by the addition of a ligase. When the ligated probes are isolated from the DNA segment, both types of labeling can be observed together, confirming the presence of the polymorphic allele-specific nucleotide sequence.

In other embodiments, alterations in electrophoretic mobility will be used to identify mutations or identify of the allelic variant of a polymorphic region. For example, single strand conformation polymorphism (SSCP) may be used to detect differences in electrophoretic mobility between mutant and wild type nucleic acids (Orita et al., PNAS USA (1989) 86: 2766, 1989). Single-stranded DNA fragments of sample and control nucleic acids are denatured and allowed to renature. Because the secondary structure of single-stranded nucleic acids varies according to sequence, the resulting alteration in electrophoretic mobility enables the detection of even a single base change. The DNA fragments may be labeled or detected with labeled probes. In one embodiment, the method utilizes heteroduplex analysis to separate double stranded heteroduplex molecules on the basis of changes in eletrophoretic mobility (Keen et al., Trends Genet. 7: 5, 1991).

In yet another embodiment, the movement of mutant or wild-type fragments in polyacrylamide gels containing a gradient of denaturant is assayed using denaturing gradient gel electrophoresis (DGGE) (Myers et al., Nature 313: 495, 1985). When DGGE is used as the method of analysis, DNA will be modified to insure that it does not completely denature, for example by adding a GC clamp of approximately 40 by of high-melting GC-rich DNA by PCR. In a further embodiment, a temperature gradient is used in place of a denaturing agent gradient to identify differences in the mobility of control and sample DNA (Rosenbaum and Reissner. Biophys. Chem. 265: 12753, 1987).

In another embodiment, identification of the allelic variant can be carried out using an OLA (U.S. Pat. No. 4,998,617; Landegren et al., Science 241: 1077, 1988). The OLA protocol uses two oligonucleotides, which are designed to be capable of hybridizing to abutting sequences of a single strand of a target. One of the oligonucleotides is linked to a separation marker, e.g., biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson et al., have described a nucleic acid detection assay that combines attributes of PCR and OLA (Nickerson et al., PNAS 87: 8923-8927, 1990). In this method, PCR is used to achieve the exponential amplification of target DNA, which is then detected using OLA. For example, U.S. Pat. No. 5,593,826 discloses an OLA using an oligonucleotide having 3′-amino group and a 5′-phosphorylated oligonucleotide to form a conjugate having a phosphoramidate linkage. In another variation, OLA combined with PCR permits typing of two alleles in a single microtiter well (Tobe et al., 24 Nucleic Acids Res. 24: 3728, 1996). By marking each of the allele-specific primers with a unique hapten, e.g., digoxigenin and fluorescein, each OLA reaction can be detected by using hapten specific antibodies that are labeled with different enzyme reporters, alkaline phosphatase, or horseradish peroxidase. This system permits the detection of the two alleles using a high throughput format that leads to the production of two different colors.

In other embodiments, an SNP can be detected by using a specialized exonuclease-resistant nucleotide (see, for example, U.S. Pat. No. 4,656,127, the contents of which are hereby incorporated by reference). According to the method, a primer complementary to the allelic sequence immediately 3′ to the polymorphic site is permitted to hybridize to the target molecule obtained from the subject or reference control cells. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identify of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amount of extraneous sequence data.

In another embodiment, a solution-based method is used for determining the identity of the nucleotide of a polymorphic site (International Publication No. WO 91/02087). A primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

In still a further embodiment, a method known as Genetic Bit Analysis (GBA™) can be used to determine genotype (International Publication No. WO 92/15712). The method uses mixtures of labeled terminators and primer that is complementary to the sequence 3′ to a polymorphic site. The labeled terminator that is incorporated is thus determined by, and complementary to, the nucleotide present in the polymorphic site of the target molecule being evaluated.

A. Primer Design

Many pharmacogenomic tests are already commercially available, and already include primers that hybridize with DNA that contains the mutations of interest. However, primers can be developed by using published sequences, including consensus sequences, to design or select primers for use in amplification of a given piece of DNA. The selection of sequences to be used to construct primers that flank a “locus of interest” can be made by examining the sequence of the loci of interest, and the surrounding base pairs. The recently published sequence of the human genome can provide a source of useful consensus sequence information from which to design primers to flank a desired human gene locus of interest.

By “flanking” a locus of interest is meant that the sequences of the primers are such that at least a portion of the 3′ region of one primer is complementary to the antisense strand of the template DNA and upstream of the locus of interest (forward primer), and at least a portion of the 3′ region of the other primer is complementary to the sense strand of the template DNA and downstream of the locus of interest (reverse primer). A “primer pair” is intended to specify a pair of forward and reverse primers. Both primers of a primer pair anneal in a manner that allows extension of the primers, such that the extension results in amplifying the template DNA in the region of the locus of interest.

In some embodiments, a plurality of mutations are present in the reference controls, so that the screening method can be tested concurrently for the ability of the reference controls to bind to a plurality of primers. For example, a plurality of reference controls for the 10 or 20 most frequently occurring mutation sites in a disease-associated gene, gene associated with poor response to a given therapeutic regimen, or metabolic enzyme can be prepared, and combined with primers that are designed to detect the majority of the disease carriers, patients with a low likelihood of responding to a given therapy, or patients with poor metabolism. Thus, the efficacy of the reference controls can be confirmed, and, once confirmed, the ability of putative primers for use in pharmacogenomic assays can also be confirmed.

B. Amplification of the “Loci of Interest” or “Hybridization Region”

The reference control (or, once the assay has been validated, a sample to be tested for having a mutation of interest) can be amplified using any suitable method known in the art including but not limited to PCR (polymerase chain reaction), 3SR (self-sustained sequence reaction), LCR (ligase chain reaction), RACE-PCR (rapid amplification of cDNA ends), PLCR (a combination of polymerase chain reaction and ligase chain reaction), Q-beta phage amplification (Shah et al., J. Medical Micro. 33: 1435-41 (1995)), SDA (strand displacement amplification), SOE-PCR (splice overlap extension PCR), and the like.

Ideally, the reference control is amplified using PCR (PCR: A Practical Approach, M. J. McPherson, et al., IRL Press (1991); PCR Protocols: A Guide to Methods and Applications, Innis, et al., Academic Press (1990); and PCR Technology: Principals and Applications of DNA Amplification, H. A. Erlich, Stockton Press (1989)). PCR is also described in numerous U.S. patents, including U.S. Pat. Nos. 4,683,195; 4,683,202; 4,800,159; 4,965.188; 4,889,818; 5,075,216; 5,079,352; 5,104,792, 5,023,171; 5,091,310; and 5,066,584.

Any DNA polymerase that catalyzes primer extension can be used including but not limited to E. coli DNA polymerase, Klenow fragment of E. coli DNA polymerase I, T7 DNA polymerase, T4 DNA polymerase, Taq polymerase, Pfu DNA polymerase, Vent DNA polymerase, or sequenase. Preferably, a thermostable DNA polymerase is used. A “hot start” PCR can also be performed wherein the reaction is heated to 95° C. for two minutes prior to addition of the polymerase or the polymerase can be kept inactive until the first heating step in cycle 1. “Hot start” PCR can be used to minimize nonspecific amplification. Any number of PCR cycles can be used to amplify the DNA, including but not limited to 2, 5, 10, 15, 20, 25, 30, 35, 40, or 45 cycles. In a most preferred embodiment, the number of PCR cycles performed is such that equimolar amounts of each loci of interest are produced.

C. Purification of Amplified DNA

It may not be necessary to purify the amplified DNA to confirm that the reference control can be detected by a particular assay. However, if purification is preferred, the 5′ end of the primer can optionally be modified with a tag that facilitates purification of the PCR products. In a preferred embodiment, the first primer is modified with a tag that facilitates purification of the PCR products. The modification is preferably the same for all primers, although different modifications can be used if it is desired to separate the PCR products into different groups.

The tag can be a radioisotope, fluorescent reporter molecule, chemiluminescent reporter molecule, antibody, antibody fragment, hapten, biotin, derivative of biotin, photobiotin, iminobiotin, digoxigenin, avidin, enzyme, acridinium, sugar, enzyme, apoenzyme, homopolymeric oligonucleotide, hormone, ferromagnetic moiety, paramagnetic moiety, diamagnetic moiety, phosphorescent moiety, luminescent moiety, electrochemiluminescent moiety, chromatic moiety, moiety having a detectable electron spin resonance, electrical capacitance, dielectric constant or electrical conductivity, or combinations thereof.

In one embodiment, the 5′ ends of the primers are biotinylated (Kandpal et al., Nucleic Acids Res. 18:1789-1795 (1990); Kaneoka et al., Biotechniques 10:30-34 (1991); Green et al., Nucleic Acids Res. 18:6163-6164 (1990)). The biotin provides an affinity tag that can be used to purify the copied DNA from the genomic DNA or any other DNA molecules that are not of interest. Biotinylated molecules can be purified using a streptavidin coated matrix, including but not limited to Streptawell, transparent, High-Bind plates from Roche Molecular Biochemicals (catalog number 1 645 692, as listed in Roche Molecular Biochemicals, 2001 Biochemicals Catalog).

The amplified DNA can also be purified using non-affinity methods known in the art, for example, by polyacrylamide gel electrophoresis using standard protocols or by gel or filter columns such as those sold by Qiagen, Whatman, and EdgeBio.

D. Digestion of Amplified DNA

The amplified DNA can be digested with a restriction enzyme that recognizes a sequence that had been provided on the primer using standard protocols known within the art.

E. Confirmation of the Binding of Putative Reference Controls to Assay Primers or Probes

The pharmacogenomic assays described above, using known primers or probes that hybridize with sequences which include a mutation of interest can verify, through binding and subsequent detection of the binding, that the reference control included a mutation of interest. Detection can be through detection of fluorescent or other labels described herein, chromatography, mass spectrometry, electrophoresis, and the like.

Once the reference controls have been identified, they can be used to screen putative primers for their use in detecting samples that include the mutation of interest.

### VII. Use of the Reference Controls in Clinical Trials

Once the reference controls are identified using the methods described herein, they can be used in clinical trials to confirm that the genetic tests used to identify patients who have poor metabolism, or who are poor responders, are working correctly.

The testing can be random, or at preselected intervals. The reference controls have ideally been pre-screened for the primers used in the genetic test. Accordingly, they should also bind in the actual genetic test used in the clinical trial. If the clinical trial fails to identify the reference controls as either positives (i.e., they include the mutation) or negatives (i.e., they do not include the mutation), then the genetic screen can be reviewed to determine why there was a failure to properly detect the reference controls.

Using the reference controls and methods described herein, clinical trial data can be made more reliable.

### VIII. Use of the Reference Controls in Patient Care

As with their use in clinical trials, once the reference controls are identified using the methods described herein, they can be used in patient care to confirm that the pharmacogenomic screens used to identify patients who have poor metabolism, or who are poor responders, are working correctly.

If the pharmacogenomic screens used to test a plurality of patients for their ability to respond to a given therapy, or to metabolize drugs correctly, fails to identify the reference controls, then the pharmacogenomic screen can be reviewed to determine why there was a failure to properly detect the reference controls. Accordingly, using the reference controls and methods described herein, patient treatment can be made more reliable.

### IX. Use of the Reference Controls in Assay Development

The reference controls can be used in assay development, where probes are developed for use in detecting the presence of various mutations. A putative probe (i.e., a probe that is believed to bind to a particular sequence containing the SNP of interest, but which has not yet been validated) can be tested for its ability to bind to, and thus detect, the mutation of interest, by using a validated reference control. Detection of probe binding to the reference control can be detected by various means known to those of skill in the art, typically involving the detection of a label on the probe. The label can be any type of label commonly used, including fluorescent probes, biotinylated probes, and the like. Once the assay has been performed, and probe binding has been verified through detection means specified by the assay, the probe becomes a validated probe.

There are several assay methods that include internal controls, for example, various DNA-chips that include a plurality of primers bound to a chip. Validated reference controls can be used, for example, as external controls, when designing the chips to provide an additional way to confirm that the chip has the ability to detect mutations of interest.

### X. Use of the Reference Controls in Laboratory Proficiency Testing

Once the reference controls and primers have been validated, they can be used by laboratory personnel to confirm that the laboratory personnel can perform the assays in a proper manner. That is, with a properly validated reference control and primer, the assay should, when performed correctly, yield results which are consistent with the reference control. If a positive control is used, the presence of the mutation should be determined. If a negative control is used, the absence of the mutation should be determined. Ideally, a combination of positive and negative controls are used, to more fully validate the laboratories' ability to detect both positive and negative samples.

As discussed above, there are several assay methods that include internal controls. Validated reference controls can be used, for example, as external controls, to confirm that the chips actually detect mutations of interest when used.

When validating that a laboratory is capable of performing the assays correctly, this can be done before individual laboratory personnel test actual samples, in a training program, or periodically, i.e., at regular or random intervals, to provide a means for continuous validation.

The present invention will be better understood with reference to the following non-limiting examples.

### Example 1

### Reference Control Cell Lines

Using the methods described herein, cell lines were generated with alleles for specific mutations in CYP450, namely, CYP2D6, CYP2C9 and CYP2C19, in addition to UGT1A1 and VKORC1. Cell lines containing mutations within CYP2A6, CYP3A4, CYP3A5, DPD, NAT2, and MDR1 are being developed. For each gene, there are numerous alleles, and Table 1, below, shows the cell lines that have been prepared using the methods described herein.

The cell lines were prepared as follows. Blood was drawn from properly consented subjects. Per the informed consent, all subjects were willing to be contacted in the future in order to provide additional blood samples. The DNA from these blood samples was extracted and purified, and each one was tested for the appropriate mutations (polymorphisms), in this case, mutations in the CYP450, VKORC1, and UGT1A1 genes. Interesting subjects were identified, and called back for additional blood samples. The lymphocytes were isolated and immortalized. Where possible, full characterization of each gene was carried out by performing bi-directional sequencing on most or all of the entire gene. For example, DNA sequencing was performed on a 6.7 kb region consisting of the CYP2D6 gene, in addition to upstream and downstream sequences.

TABLE 1
REFERENCE CONTROL CELL LINES
Cell Line CL0001
CYP2D6

| | |
|---|---|
| Product Name(s) | CYP 2D6 *4A/*2AxN |
| CYP2D6 | *4A(100C > T, 974C > A, 984A > G, 997C > G, 1661G > C, 1846G > A, |
| Mutations1 | 4180G > C); *2AxN (CYP2D6 duplication, |
| | −1584C > G, −740C > T, −678G > A, 2D7 conversion in intron 2, 214G > C, 221C > A, |
| | 223C > G, 227T > C, 232G > C, 233A > C, 245A > G, 1661G > C, 2850C > T, 4180G > C), |
| | additional mutations (−1770G > A, 310G > T; 746C > G; 843T > G; 3384A > C; |
| | 3584G > A; 3790C > T, 4401C > T, 4481G > A, 4656-58delACA, −1426C > T, 310G > T, |
| | 746C > G, 843T > G, 3384A > C, 4481G > A) |

CYP2C9

| | |
|---|---|
| Product Name(s) | CYP2C9 *1A/*1^{N} |
| CYP2C9 | *1A (none); *1^{N}(251T > C, 3411T > C, 33658A > G, 50056A > T) |
| Mutations² | |

VKORC1

| | |
|---|---|
| Product Name(s)³ | VKORC1 (GGCCAA) |
| VKORC1 | Haplotype 1 (3730G > A); Haplotype 2 (3730G > A) |
| Mutations⁴ | |

UGT1A1

| | |
|---|---|
| Product Name(s)⁵ | UGT1A1 *28/wt |
| UGT1A1 | *28[A(TA)₇TAA]; wt [A(TA)₆TAA] |
| Mutations^{5,6} | |

CYP2C19

| | |
|---|---|
| Product Name(s)⁷ | CYP2C19 wt/wt |
| CYP2C19 | wt (none); wt(none) |
| Mutations^{7,8} | |

Cell Line CL0002
CYP2D6

| | |
|---|---|
| Product Name(s) | CYP2D6 *29/*2AxN |
| CYP2D6 | *29(1659G > A, 1661G > C, 2850C > T, 3183G > A, 4180G > C); *2AxN(CYP2D6 |
| Mutations¹ | duplication, |
| | −1584C > G, −740C > T, −678G > A, 2D7 conversion in intron 2, 214G > C, 221C > A, |
| | 223C > G, 227T > C, 232G > C, 233A > C, 245A > G, 1661G > C, 2850C > T, 4180G > C); |
| | additional mutations (−1770G > A, 310G > T, 3384A > C, 3584G > A, 3790C > T, |
| | 4481G > A, 4656-58delACA, −740C > T, −678G > A) |

CYP2C9

| | |
|---|---|
| Product Name(s) | CYP2C9 *1A/*1^{N} |
| CYP2C9 | *1A (none); *1^{N}(−981G > A, 3856G > A, 8763C > T, 9032G > C, 10311A > G, |
| Mutations² | 33349A > G, 50056A > T) |

VKORC1

| | |
|---|---|
| Product Name(s)³ | VKORC1 (GGCCAA) |
| VKORC1 | Haplotype 1 (3730G > A); Haplotype 2 (3730G > A) |
| Mutations⁴ | |

UGT1A1

| | |
|---|---|
| Product Name(s)⁵ | UGT1A1 wt/wt |
| UGT1A1 | wt[A(TA)₆TAA]; wt [A(TA)₆TAA] |
| Mutations^{5,6} | |

CYP2C19

| | |
|---|---|
| Product Name(s)⁷ | CYP2C19 *2/wt |
| CYP2C19 | *2(19154G > A); wt (none¹) |
| Mutations^{7,8} | |

Cell line CL003
CYP2D6

| | |
|---|---|
| Product Name(s) | CYP2D6 *2M/*17 |
| CYP2D6 | *2M(−740C > T, 2D7 conversion in intron 2, 214G > C, 221C > A, 223C > G, 227T > C, |
| Mutations¹ | 232G > C, 233A > C, 245A > G, 310G > T, 746C > G, 843T > G, 1661G > C, 2850C > T, |
| | 3384A > C, 3584G > A, 3790C > T, 4180G > C); *17(1023C > T, 1661G > C, 2850C > T, |
| | 4180G > C); additional mutations (−1298G > A, 4656-58delACA, −740C > T, 310G > T, |
| | 843T > G, 3384A > C, 3584G > A, 3790C > T) |

CYP2C9

| | |
|---|---|
| Product Name(s) | CYP2C9 *1^{N}/*1^{N} |
| CYP2C9 | *1^{N} (50056A > T); *1^{N}(−981G > A, 251T > C, 3411T > C, 33658A > G, 50056A > T) |
| Mutations² | |

VKORC1

| | |
|---|---|
| Product Name(s)³ | VKORC1 (GGCCGA) |
| VKORC1 | Haplotype 1 (3730G > A); Haplotype 2 (none) |
| Mutations⁴ | |

UGT1A1

| | |
|---|---|
| Product Name(s)⁵ | UGT1A1 *37/wt |
| UGT1A1 | *28[A(TA)₈TAA]; wt [A(TA)₆TAA] |
| Mutations^{5,6} | |

CYP2C19

| | |
|---|---|
| Product Name(s)⁷ | CYP2C19 wt/wt |
| CYP2C19 | wt (none); wt (none |
| Mutations^{7,8} | |

Cell Line CL0004
CYP2D6

| | |
|---|---|
| Product Name(s) | CYP2D6 *35/*41 |
| CYP2D6 Mutations¹ | *35(−1584C > G, 31G > A, 1661G > C, 2850C > T, 4180G > C); *41(−740C > T, −678G > |
| | A, 2D7 conversion in intron 2, 214G > C, 221C > A, 223C > G, 227T > C, |
| | 232G > C, 233A > C, 245A > G, 1661G > C, 2850C > T, 2988G > A, 4180G > C); |
| | additional mutations (310G > T, 746C > G, 843T > G, 3384A > C, 3790C > T, |
| | 4481G > A, 4656-58delACA) |

CYP2C9

| | |
|---|---|
| Product Name(s) | CYP2C9 *1A/*1^{N} |
| CYP2C9 Mutations² | *1A (none); *1^{N}(−3089G > A, −1188T > C, 3898C > T, 3924T > C, 47639C > T, |
| | 50056A > T) |

VKORC1

| | |
|---|---|
| Product Name(s)³ | VKORC1 (GGCCAA) |
| VKORC1 | Haplotype 1 (3730G > A); Haplotype 2 (3730G > A) |
| Mutations⁴ | |

UGT1A1

| | |
|---|---|
| Product Name(s)⁵ | UGT1A1 wt/wt |
| UGT1A1 | wt [A(TA)₆TAA]; wt [A(TA)₆TAA] |
| Mutations^{5,6} | |

CYP2C19

| | |
|---|---|
| Product Name(s)⁷ | CYP2C19 *2/wt |
| CYP2C19 | *2(19154G > A); wt (none¹) |
| Mutations^{7,8} | |

Cell Line CL005
CYP2D6

| | |
|---|---|
| Product Name(s) | CYP2D6 *1A/*9 |
| CYP2D6 Mutations¹ | *1A (none); *9(2613-2615delAGA) |

CYP2C9

| | |
|---|---|
| Product Name(s) | CYP2C9 *1A/*1^{N} |
| CYP2C9 Mutations² | *1A (none); *1^{N}(−981G > A, 251T > C, 3411T > C, 33658A > G, 50056A > T) |

VKORC1

| | |
|---|---|
| Product Name(s)³ | VKORC1 (GGCCGG) |
| VKORC1 | Haplotype 1(none); Haplotype 2(none) |
| Mutations⁴ | |

UGT1A1

| | |
|---|---|
| Product Name(s)⁵ | UGT1A1 wt/wt |
| UGT1A1 | wt⁵ [A(TA)₆TAA]; wt⁵ [A(TA)₆TAA] |
| Mutations^{5,6} | |

CYP2C19

| | |
|---|---|
| Product Name(s)⁷ | CYP2C19 wt/wt |
| CYP2C19 | wt (none); wt (none) |
| Mutations^{7,8} | |

Cell Line CL006
CYP2D6

| | |
|---|---|
| Product Name(s) | CYP2D6 *3A/*4A |
| CYP2D6 Mutations¹ | *3A(2549A > del); *4A(100C > T, 974C > A; 984A > G, 997C > G, 1661G > C, |
| | 1846G > A, 4180G > C); additional mutations (−1426C > T, 310G > T, 746C > G, |
| | 843T > G, 3384A > C, 4401C > T) |

CYP2C9

| | |
|---|---|
| Product Name(s) | CYP2C9 *1^{N}/*3B |
| CYP2C9 Mutations² | *1^{N}(251T > C, 3411T > C, 33658A > G, 50056A > T); *3B(−1911T > C, −1885C > G, −1537G > |
| | A, −1188T > C, 3856G > A, 3924T > C, 8763C > T, 33349A > G, 42614A > C, |
| | 47545A > T, 50053G > A, 50056A > T, 50298A > T, 50742T > A) |

VKORC1

| | |
|---|---|
| Product Name(s)³ | VKORC1 (GGCCAA) |
| VKORC1 | Haplotype 1 (3730G > A); Haplotype 2 (3730G > A) |
| Mutations⁴ | |

UGT1A1

| | |
|---|---|
| Product Name(s)⁵ | UGT1A1 *28/28 |
| UGT1A1 | *28[A(TA)₇TAA]; *28[A(TA)₇TAA] |
| Mutations^{5,6} | |

CYP2C19

| | |
|---|---|
| Product Name(s)⁷ | CYP2C19 wt/wt |
| CYP2C19 | wt (none); wt (none) |
| Mutations^{7,8} | |

Cell Line CL007
CYP2D6

| | |
|---|---|
| Product Name(s) | CYP2D6 *1A/*6B |
| CYP2D6 | *1A (none); *6B(1707T > del, 1976G > A) |
| Mutations¹ | |

CYP2C9

| | |
|---|---|
| Product Name(s) | CYP2C9 *2A/*3B |
| CYP2C9 | *2A(−1188T > C, −1096A > G, −620G > T, −485T > A, −484C > A, 3608C > T, 3856G > A, |
| Mutations² | 8763C > T, 33349A > G, 50053G > A, 50056A > T); *3B(−1911T > C, −1885C > G, −1537G > |
| | A, −1188T > C, 3856G > A, 3924T > C, 8763C > T, 33349A > G, 42614A > C, |
| | 47545A > T, 50053G > A, 50056A > T, 50298A > T, 50742T > A) |

VKORC1

| | |
|---|---|
| Product Name(s)³ | VKORC1 (GACTGG) |
| VKORC1 | Haplotype 1(−1639G > A, 1173C > T); Haplotype 2(none) |
| Mutations⁴ | |

UGT1A1

| | |
|---|---|
| Product Name(s)⁵ | UGT1A1 *28/*36 |
| UGT1A1 | *28[A(TA)₇TAA]; *36[A(TA)₅TAA]; |
| Mutations^{5,6} | |

CYP2C19

| | |
|---|---|
| Product Name(s)⁷ | CYP2C19 wt/wt |
| CYP2C19 | wt (none); wt (none) |
| Mutations^{7,8} | |

Cell Line CL008
CYP2D6

| | |
|---|---|
| Product Name(s) | CYP2D6 *6B/*41 |
| CYP2D6 Mutations¹ | *6B (1707T > del, 1976G > A); *41 (−740C > T, −678G > A, 2D7 conversion in intron |
| | 2, 214G > C, 221C > A, 223C > G, 227T > C, 232G > C, 233A > C, 245A > G, 1661G > C, |
| | 2850C > T, 2988G > A, 4180G > C, additional mutations (310G > T, 746C > G, |
| | 843T > G, 3384A > C, 3790C > T, 4481G > A, 4656-58delACA) |

CYP2C9

| | |
|---|---|
| Product Name(s) | ND |
| CYP2C9 Mutations² | ND |

VKORC1

| | |
|---|---|
| Product Name(s)³ | VKORC1 (GGCCGA) |
| VKORC1 | Haplotype 1(3730G > A); Haplotype 2(none) |
| Mutations⁴ | |

UGT1A1

| | |
|---|---|
| Product Name(s)⁵ | UGT1A1 *28/wt |
| UGT1A1 | *28[A(TA)₇TAA]; wt [A(TA)₆TAA]; |
| Mutations^{5,6} | |

CYP2C19

| | |
|---|---|
| Product Name(s)⁷ | CYP2C19 wt/wt |
| CYP2C19 | wt (none); wt (none) |
| Mutations^{7,8} | |

Cell Line CL009
CYP2D6

| | |
|---|---|
| Product Name(s) | CYP2D6 *1^{N}/*5 |
| CYP2D6 Mutations¹ | *1^{N} (125G > A, 1495T > C); *5 (CYP2D6 gene deletion on a single chromosome) |

CYP2C9

| | |
|---|---|
| Product Name(s) | CYP2C9 *1A/*1^{N} |
| CYP2C9 Mutations² | *1A (none); *1^{N}(−1188T > C, −981G > A, 3856G > A, 8763C > T, 9032G > C, |
| | 10311A > G, 33349A > G, 50056A > T) |

VKORC1

| | |
|---|---|
| Product Name(s)³ | VKORC1 (AATTGG) |
| VKORC1 | Haplotype 1(−1639G > A, 1173C > T); Haplotype 2(−1639G > A, 1173C > T) |
| Mutations⁴ | |

UGT1A1

| | |
|---|---|
| Product Name(s)⁵ | UGT1A1 wt/wt |
| UGT1A1 | wt⁵ [A(TA)₆TAA]; wt⁵ [A(TA)₆TAA] |
| Mutations^{5,6} | |

CYP2C19

| | |
|---|---|
| Product Name(s)⁷ | CYP2C19 *3/wt |
| CYP2C19 | *3(17948G > A); wt⁷ (none) |
| Mutations^{7,8} | |

Cell Line CL0010
CYP2D6

| | |
|---|---|
| Product Name(s) | CYP2D6 *5/*41 |
| CVP2D6 Mutations¹ | *5 (CYP2D6 gene deletion on one chromosome); *41 (−1235A > G, −740C > T, −678G > |
| | A, 2D7 conversion in intron 2, 214G > C, 221C > A, 223C > G, 227T > C, |
| | 232G > C, 233A > C, 245A > G, 1661G > C, 2850C > T, 2988G > A, 4180G > C, |
| | additional mutations (310G > T, 746C > G, 843T > G, 3384A > C, 3790C > T, |
| | 4481G > A, 4656-58delACA) |

CYP2C9

| | |
|---|---|
| Product Name(s) | CYP2C9 *1A/*1^{N} |
| CYP2C9 Mutations² | *1A (none); *1^{N}(−981G > A) |

VKORC1

| | |
|---|---|
| Product Name(s)³ | VKORC1 (GGCCGA) |
| VKORC1 | Haplotype 1 (3730 G > A); Haplotype 2 (none) |
| Mutations⁴ | |

UGT1A1

| | |
|---|---|
| Product Name(s)⁵ | UGT1A1 wt/wt |
| UGT1A1 | wt⁵ [A(TA)₆TAA]; wt⁵ [A(TA)₆TAA] |
| Mutations^{5,6} | |

CYP2C19

| | |
|---|---|
| Product Name(s)⁷ | CYP2C19 wt/wt |
| CYP2C19 | wt (none); wt (none) |
| Mutations^{7,8} | |

Cell Line CL0011
CYP2D6

| | |
|---|---|
| Product Name(s) | CYP2D6 *5/*5 |
| CYP2D6 Mutations¹ | *5(CYP2D6 gene deletion on a single chromosome); *5(CYP2D6 gene deletion on |
| | a single chromosome) |

CYP2C9

| | |
|---|---|
| Product Name(s) | CYP2C9 *1A/*1^{N} |
| CYP2C9 Mutations² | *1A (none); *1^{N}(−981G > A) |

VKORC1

| | |
|---|---|
| Product Name(s)³ | VKORC1 (GACTGG) |
| VKORC1 | Haplotype 1(−1639G > A, 1173C > T); Haplotype 2(none) |
| Mutations⁴ | |

UGT1A1

| | |
|---|---|
| Product Name(s)⁵ | UGT1A1 wt/wt |
| UGT1A1 | wt [A(TA)₆TAA]; wt [A(TA)₆TAA] |
| Mutations^{5,6} | |

CYP2C19

| | |
|---|---|
| Product Name(s)⁷ | CYP2C19 wt/wt |
| CYP2C19 | wt (none); wt (none) |
| Mutations^{7,8} | |

Cell Line CL0015
CYP2D6

| | |
|---|---|
| Product Name(s) | CYP2D6 *4A/*7 |
| CYP2D6 Mutations¹ | *4A(100C > T, 974C > A; 984A > G, 997C > G, 1661G > C, 1846G > A, 4180G > C); *7 |
| | (2935A > C); additional mutations (−1426C > T, −1235A > G, 310G > T, 746C > G, |
| | 843T > G, 3384A > C, 4401C > T) |

CYP2C9

| | |
|---|---|
| Product Name(s) | CYP2C9 *2A/*2A |
| CYP2C9 Mutations² | *2A(−1188T > C, −1096A > G, −620G > T, −485T > A, −484C > A, 3608C > T, 3856G > A, |
| | 8763C > T, 33349A > G, 50053G > A, 50056A > T); *2A(−1188T > C, −1096A > G, −620G > |
| | T, −485T > A, −484C > A, 3608C > T, 3856G > A, 8763C > T, 33349A > G, |
| | 50053G > A, 50056A > T) |

VKORC1

| | |
|---|---|
| Product Name(s)³ | ND |
| VKORC1 | ND |
| Mutations⁴ | |

UGT1A1

| | |
|---|---|
| Product Name(s)⁵ | ND |
| UGT1A1 | ND |
| Mutations^{5,6} | |

CYP2C19

| | |
|---|---|
| Product Name(s)⁷ | ND |
| CYP2C19 | ND |
| Mutations^{7,8} | |

Cell Line CL0016
CYP2D6

| | |
|---|---|
| Product Name(s) | CYP2D6 *1A/*1A |
| CYP2D6 Mutations¹ | *1A (none); *1A (none) |

CYP2C9

| | |
|---|---|
| Product Name(s) | CYP2C9 *1A/*1^{N} |
| CYP2C9 Mutations² | *1A (none); *1^{N}(−981G > A, 251T > C, 3411T > C, 33658A > G, 50056A > T) |

VKORC1

| | |
|---|---|
| Product Name(s)³ | ND |
| VKORC1 Mutations⁴ | ND |

UGT1A1

| | |
|---|---|
| Product Name(s)⁵ | ND |
| UGT1A1 | ND |
| Mutations^{5,6} | |

CYP2C19

| | |
|---|---|
| Product Name(s)⁷ | ND |
| CYP2C19 | ND |
| Mutations^{7,8} | |

¹= Nucleotide changes are based on the “wild-type” CYP2D6 gene sequences (accession numbers M33388 and AY545216 with the A of the translational start codon defined as +1). Allelic designations were determined based on data reported by the Human Cytochrome P450 Nomenclature Committee (http://www.imm.ki.se/CYPalleles/cyp2d6.htm).
²= Nucleotide changes are based on the “wild-type” CYP2C9 gene sequence (accession number AL359672 with the A of the translational start codon defined as +1). Allelic designations were determined based on data reported by the Human Cytochrome P450 Nomenclature Committee (http://www.imm.ki.se/CYPalleles/cyp2c9.htm).
³= VKORC1 product name depicts biallelic genotypes at genomic positions −1639 (XX----), 1173 (--XX--) and 3730 (----XX) (e.g. for the product name VKORC1 (AATTGG), the genotypes are −1639AA, 1173TT, and 3730GG).
⁴= Nucleotide changes are based on the “wild-type” VKORC1 gene sequence (accession number AY587020 with the A of the translational start codon defined as +1).
⁵= “Wild-type” genotype indicates that UGT1A1 *28, *36, and *37 are not present in the cell line. Testing for additional UGT1A1 alleles was not performed.
⁶= Nucleotide changes are based on the “wild-type” UGT1A1 gene sequence (accession number AF297093 with the A of the translational start codon defined as +1). The number of TA repeats (5-8 repeats depicted by a subscript) for each allele is listed with adjacent sequence. Allelic designations were determined based on data reported by the UGT Nomenclature Committee (http://galien.pha.ulaval.ca/alleles/alleles.html).
⁷= “Wild-type” genotype indicates that CYP2C19 *2, *3, *4, *5, and *10 are not present in the cell line. Testing for additional CYP2C19 alleles was not performed.
⁸= Nucleotide changes are based on the “wild-type” CYP2C19 gene sequence (accession number NT_030059 with the A of the translational start codon defined as +1). Allelic designations were determined based on data reported by the Human Cytochrome P450 Nomenclature Committee (http://www.imm.ki.se/CYPalleles/cyp2c19.htm).
N = novel polymorphisms
ND = not determined

The individual cell lines described above in this Example, the human genomic DNA derived from these cells, and the uses of the genomic DNA in clinical trials, patient treatment, assay validation, and assay development are all within the scope of the invention.

Applicants have deposited cells according to the invention with the American Type Culture Collection (ATCC), Manassas. VA. Accession Number corresponding to the cell line designation used herein are as follows: ATCC PTA-9083 for CL001, ATCC PTA-9084 for CL002, ATCC PTA-9085 for CL003, ATCC PTA-9086 for CL004, ATCC PTA-9087 for CL005, ATCC PTA-9088 for CL006, ATCC PTA-9089 for CL007, ATCC PTA-9090 for CL008, ATCC PTA-9091 for CL009, ATCC PTA-9092 for CL0010, ATCC PTA-9093 for CL011, ATCC PTA-9097 for CL015, and ATCC PTA-9098 for CL016. The cells deposited with the ATCC were taken from the same deposit maintained by Gentris Corporation, Morrisville, N.C., USA since prior to the filing date of this application. The deposits of the cells will be maintained without restriction in the ATCC depository for a period of 30 years, or 5 years after the most recent request, or for the effective life of the patent, whichever is longer, and will be replaced if the deposit becomes non-viable during that period.

### Example 2

### Genomic DNA Containing a Mutation of Interest and a Mutation Preventing Binding to a Desired Primer

A series of screens were conducted to find samples that include a mutation of interest, such as a mutation in CYP450; but the primer of interest was unable to detect the presence or absence of such a mutation. For example, a sample was screened for a mutation in CYP450, the *6 mutation. A proprietary assay which detects *6 showed no band for wildtype or mutant. Initially, it was believed that there was a mutation in the primer binding site that prevented the sample from being properly identified for its *6 status. A careful analysis of the sample showed that there was, indeed, a mutation in the binding site. Because of this second mutation, the sample would not function as a suitable reference control when used with that specific primer to detect the specific *6 mutation but was suitable as a reference control to detect possible mutations in the primer binding site for *6. In a second example, it was demonstrated that a control that was characterized as CYP2D6*1/*5 failed to give a signal when testing for CYP2D6*12 on certain platforms, such as the Tm BioSciences Tag-It™ assay and the ThirdWave Invader® assay. Upon re-examination of the resequencing data it was discovered that there is a mutation near the *12 site that prevents some primers from binding. This sample is suitable as a reference control for the CYP2D6*1/*5 but would not be a good control for testing the performance of the *12 assay.

The foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

1-54. (canceled)

55. A method of clinical pharmacogenomic screening comprising:
a) screening a sample for the presence of one or more mutations in one or more Cytochrome P450 (CYP450) genes or other gene associated with drug metabolism, wherein the presence of the one or more mutations is indicative of a patient with altered metabolism; and
b) including a reference control in a random or predetermined manner in the screening, wherein the reference control comprises DNA comprising the mutations indicative of a patient with altered metabolism,
wherein the detection of the presence of one or more mutations in one or more drug-metabolizing genes in the reference control verifies that the screening is effective to detect the same one or more mutations in one or more drug-metabolizing genes in the sample.

56. The method of claim 55, wherein the one or more mutations in one or more of the drug-metabolizing genes is in a gene selected from the group consisting of CYP2D6, CYP2C19, CYP2C9, CYP2C8, and CYP3A5, CYP3A4, CYP2A6, CYP2B6, UGT1A1, DPD, ERCC15 MDR1, ADH2, VKORC1, NAT1, and NAT2.

57. The method of claim 55, wherein the one or more mutations in one or more of the drug-metabolizing genes is in a gene selected from the group consisting of CYP2D6, CYP2C19, CYP2C9, UGT1A1, and VKORC1.

58. The method of claim 55, wherein the one or more mutations in one or more of the drug-metabolizing genes is in VKORC1.

59. The method of claim 55, wherein the reference control comprises one or more cells from a cell line, wherein the cell line is selected from the group consisting of cell line CL001 (ATCC Accession No. PTA-9083), CL002 (ATCC Accession No. PTA-9084), CL003 (ATCC Accession No. PTA-9085), CL004 (ATCC Accession No. PTA-9086), CL005 (ATCC Accession No. PTA-9087), CL006 (ATCC Accession No. PTA-9088), CL007 (ATCC Accession No. PTA-9089), CL008 (ATCC Accession No. PTA-9090), CL009 (ATCC Accession No. PTA-9091), CL010 (ATCC Accession No. PTA-9092), CL011 (ATCC Accession No. PTA-9093), CL015 (ATCC Accession No. PTA-9097), and CL016 (ATCC Accession No. PTA-9098).

60. A method of personalized medical therapy, comprising:
a) performing the method of screening of claim 1 on samples from a target patient population to identify patients with a genetic profile comprising one or more mutations in one or more Cytochrome P450 (CYP450) genes or other gene associated with drug metabolism;
b) treating patients identified in step a) as possessing a particular genetic profile with a therapy of interest particular to the identified genetic profile.

61. The method of claim 55, wherein the one or more mutations in one or more of the drug-metabolizing genes is in a gene selected from the group consisting of CYP2D6, CYP2C19, CYP2C9, CYP2C8, and CYP3A5, CYP3A4, CYP2A6, CYP2B6, UGT1A1, DPD, ERCC15 MDR1, ADH2, VKORC1, NAT1, and NAT2.

62. The method of claim 55, wherein the one or more mutations in one or more of the drug-metabolizing genes is in a gene selected from the group consisting of CYP2D6, CYP2C19, CYP2C9, UGT1A1, and VKORC1.

63. The method of claim 55, wherein the one or more mutations in one or more of the drug-metabolizing genes is in VKORC1.

64. The method of claim 60, wherein the genetic profile is indicative of a patient with altered metabolism.

65. The method of claim 64, wherein the altered metabolism is selected from the group consisting of: poor metabolizer, intermediate metabolizer, extensive metabolizer, and ultra-rapid metabolizer.

66. The method of claim 60, wherein the genetic profile is indicative of the effectiveness of the therapy of interest in the patient.

67. The method of claim 60, wherein the genetic profile is indicative of a patient with a genetic disorder.

68. The method of claim 60, wherein the genetic profile is indicative of a patient who should not be treated with a particular therapy.

69. The method of claim 60, wherein the therapy of interest is used to treat a disease or disorder selected from the group consisting of: cancer, heart disease, neurological disorders, psychiatric disorders, autoimmune disorders, and metabolic disorders.

70. The method of claim 60, wherein the one or more mutations comprises a mutation in VKORC1 and wherein the therapy of interest comprises administration of R-warfarin to the patient.

71. The method of claim 70, wherein the reference control in the method of screening comprises one or more cells from a cell line, wherein the cell line is selected from the group consisting of cell line CL001 (ATCC Accession No. PTA-9083), CL002 (ATCC Accession No. PTA-9084), CL003 (ATCC Accession No. PTA-9085), CL004 (ATCC Accession No. PTA-9086), CL006 (ATCC Accession No. PTA-9088), CL007 (ATCC Accession No. PTA-9089), CL008 (ATCC Accession No. PTA-9090), CL009 (ATCC Accession No. PTA-9091), CL010 (ATCC Accession No. PTA-9092), and CL011 (ATCC Accession No. PTA-9093).
